(19) United States
(12) Patent Application Publication
(10) Pub. No.: US 2025/0170293 A1
(43) Pub. Date: May 29, 2025

(54) SYSTEMS AND METHODS FOR DISTRIBUTING IRRADIATION FOR DISINFECTION

(71) Applicant: Framework Product Development LLC, Lakesode, CA (US)

(72) Inventor: Sam Rhea Sarcia, Lakeside, CA (US)

(73) Assignee: Framework Product Development LLC, Lakesode, CA (US)

(21) Appl. No.: 18/841,682

(22) PCT Filed: Apr. 21, 2023
(86) PCT No.: PCT/US2023/019424

§ 371 Date: Aug. 27, 2024

Related U.S. Application Data
(60) Provisional application No. 63/333,990, filed on Apr. 22, 2022.
(60) Provisional application No. 63/453,752, filed on Mar. 21, 2023.

(51) Int. Cl. A61L 9/20; A61L 2/10; C02F 1/32

(52) U.S. Cl. CPC A61L 9/20; A61L 2/10; C02F 1/325; A61L 2202/11; A61L 2209/12; C02F 2201/3222; C02F 2201/3228; C02F 2303/04

(57) ABSTRACT

In an embodiment, a device for controlling the direction of light from a light source is disclosed including: a first reflector having a first focal point; a light source positioned proximate to the first focal point of the first reflector; wherein the light source provides light to the first reflector from a first position having a beam angle of 180 degrees or less and wherein the first reflector reflects the light in two or more substantially collimated rays such that the two or more collimated rays are substantially parallel to each other in a first output pattern.

### CROSS-REFERENCE TO RELATED APPLICATIONS

The present application is related to Ser. No. 17/472,539, entitled “Room Disinfection Systems Comprising Concentrated Light Sources” filed Sep. 10, 2021, which is incorporated herein by reference in its entirety.

The present application claims the benefit of U.S. provisional patent application No. 63/333,990, entitled “Methods for Distributing and Applying Germicidal Irradiation to Disinfect Air” filed Apr. 22, 2022, and application No. 63/453,752, entitled “Method for Air Disinfection” filed Mar. 21, 2023, which are incorporated herein by reference in their entireties.

### TECHNICAL FIELD

The present disclosure is generally related to reflecting systems, more particularly to reflecting systems utilizing a light source to irradiate fluid for disinfection.

### BACKGROUND

It is well understood that many respiratory diseases such as SARS-CoV2, Influenza, Tuberculosis, and Rhinovirus are spread through the generation, transmission, and inhalation of aerosol particles in shared indoor spaces. The Wells-Riley equation demonstrates that this transmission mechanism may be mitigated through the implementation of ventilation interventions such as fresh air dilution, filtration, and germicidal inactivation. In the case of germicidal inactivation, radiation in the ultraviolet spectrum is applied to the air in the space where it disrupts the genetic reproduction of any biological pathogens in said air, rendering them less capable of infecting susceptible individuals.

When designing and implementing a system to provide ventilation for the purpose of reducing infection risk, it is desirable to minimize the overall costs relative to efficacy and convenient to quantify said efficacy in terms of equivalent diluting fresh air provided. Because of the exponential decay behavior of airborne pathogens exposed to germicidal irradiations, the way in which a given amount of optical power is applied to the air in the room is highly dependent on the spatial distribution of said power, and its interaction with air currents in the space being disinfected. Also, because there is a health hazard to humans, animals, and plants from the exposure to germicidal irradiation, and many surfaces will degrade it is necessary to control how any germicidal light source interacts with room contents in addition to how it is applied to the air within said room.

There are several sources of germicidal irradiation used in practice. Historically, low pressure mercury lamps emitting 254 nm light have been the predominant source utilized. Recent technological developments have enabled light emitting diodes producing germicidal wave lengths to be manufactured cost effectively with useful lifetimes. The concentrated nature of such sources, as compared to mercury lamps where radiation is emitted across the relatively large surface of the lamp, enable the utilization of novel optical systems to augment the way in which light is distributed through space.

In practice, germicidal light sources are often deployed within occupied spaces in order to inactivate potential pathogens exhaled by room occupants and limit the risk of those pathogens infecting other susceptible room occupants. The reduction in pathogen concentration achieved by a germicidal light source may be quantified in terms of the diluting clean air that would achieve an equivalent reduction in pathogen concentration. The greater the clean air rate per person, the greater the reduction in risk. The potential clean air capacity of a germicidal system is known to scale with the irradiant intensity across the volume being irradiated. And the irradiant intensity across the volume achieved by a given amount of optical power is known to scale with the length that light rays from the optical source travel with the volume before being attenuated.

The extent to which irradiant intensity across a volume produced by a light source achieves its potential for disinfection depends on how uniformly the light energy is distributed to the air in the volume. The more uniformly the irritant intensity is distributed within the volume, and the more mixing there is, the more a system will reach it's potential.

Because there is a human health hazard from some UV optical sources, it is often desirable to contain the irradiant intensity to an area where room air may pass through but where room occupants will not be exposed to the light. Because of the divergent nature of light, these often become competing priorities with the most convenient way to contain light, physical barriers, inherently constraining the transport of room air through the irradiated zone.

### SUMMARY

A device for controlling the direction of light from a light source and methods of using the device are described herein. The device may effectively disinfect fluids and may be manufactured and sold at a lower cost than commercially available irradiation systems.

In a first aspect, a device for controlling the direction of light from a light source is disclosed including: a first reflector having a first focal point and a light source positioned proximate to the first focal point of the first reflector, wherein the light source provides light to the first reflector from a first position having a beam angle of 180 degrees or less and wherein the first reflector reflects the light in two or more substantially collimated rays such that the two or more collimated rays are substantially parallel to each other in a first output pattern.

In an embodiment of the first aspect, the first reflector includes an axially symmetric parabolic or paraboloidic reflector. In an embodiment of the first aspect, the first reflector includes a specular reflection of greater than or equal to 40%. In an embodiment of the first aspect, the first reflector includes a specular reflection of greater than or equal to 80%.

In an embodiment of the first aspect, the first reflector includes a body constructed from plastic, ceramic, or metal. In an embodiment of the first aspect, the first reflector is formed from a reflective aluminum sheet that holds the shape of the reflector. In an embodiment of the first aspect, the first reflector is formed from a reflective sheet of aluminum and is supported by the reflector body. In an embodiment of the first aspect, the first reflector includes a first reflective surface, the first reflective surface including a thin film coating applied to at least a portion of the first reflective surface. In an embodiment of the first aspect, the thin film coating includes aluminum, silver, gold, or combinations thereof. In an embodiment of the first aspect, the thin film coating is applied using a metallization process selected from the group consisting of: photo-vapor-deposition, flame spraying, electroplating, and two-part silvering. In an embodiment of the first aspect, the thin film coating has a thickness between approximately 0.05 um and 5 um.

In an embodiment of the first aspect, the light source is selected from the group including light emitting diodes (LEDs), a low pressure mercury lamp, a high pressure mercury lamp, an amalgam lamp, an excimer lamp, and combinations thereof. In an embodiment of the first aspect, the light source includes one or more LEDs, the one or more LEDs emitting ultraviolet (UV) light between 180 nm and 415 nm. In an embodiment of the first aspect, the light source further includes one or more lamps operating in the visible light spectrum.

In an embodiment of the first aspect, the light source is positioned proximate to the first focal point of the first reflector via a mounting element. In an embodiment of the first aspect, the mounting element is selected from the group including one or more arms, one or more lenses, or combinations thereof. In an embodiment of the first aspect, the mounting element includes an arm that extends from a first position of the first reflector to a second position of the first reflector, the first and second positions being approximately 180 degrees apart and proximate to an edge of the first reflector. In an embodiment of the first aspect, the first reflector is at least partially surrounded by a reflector housing having one or more slots and wherein the mounting element is received by the one or more slots. In an embodiment of the first aspect, the mounting element is configured to occlude less than 15% of reflected light.

In an embodiment of the first aspect, the mounting element is further configured to remove heat from the light source, thereby functioning as a heat sink. In an embodiment of the first aspect, the arm includes a section of high thermal conductivity material.

In an embodiment of the first aspect, the arm further includes one or more heat pipes. In an embodiment of the first aspect, the mounting element includes a metal-core printed circuit board with an integrated heat sink. In an embodiment of the first aspect, the mounting element includes one or more transparent or translucent lens. In an embodiment of the first aspect, the lens is constructed from SiO₂ or Al₂O₃ and is transmissive to UV light. In an embodiment of the first aspect, a transparent or translucent lens is positioned between the light source and reflector and the surround environment. In an embodiment of the first aspect, the lens seals off a volume between the first reflector and the light source.

In an embodiment of the first aspect, the light source is movable along an axis relative to the first reflector, wherein moving the light source to a second position results in a controlled degree of divergence of the reflected light pattern. In an embodiment of the first aspect, the divergence is less than approximately 10 degrees. In an embodiment of the first aspect, the light source movement is automated. In an embodiment of the first aspect, the light source comprises a lighting element length, wherein the first reflector comprises a diameter, and wherein the lighting element length is approximately between 0.002 and 0.01 times the diameter of the first reflector.

In an embodiment of the first aspect, the device further includes a reflector housing, the reflector housing including a base portion and a circuit board. In an embodiment of the first aspect, the device further includes a power source, the power source in communication with the light source and/or the circuit board.

In an embodiment of the first aspect, the device further includes a second reflector, wherein the second reflector receives the first output pattern from the first reflector and redirects a portion of the two or more collimated rays into a second output pattern. In an embodiment of the first aspect, the second output pattern has a different central direction and/or divergent characteristics than the first output pattern. In an embodiment of the first aspect, the second reflector is conical in shape. In an embodiment of the first aspect, the second reflector has a shape that is adjustable.

In an embodiment of the first aspect, a system includes a printed circuit board; and two or more devices including a first reflector having a first focal point and a light source positioned proximate to the first focal point of the first reflector, wherein the light source provides light to the first reflector from a first position having a beam angle of 180 degrees or less and wherein the first reflector reflects the light in two or more substantially collimated rays such that the two or more collimated rays are substantially parallel to each other in a first output pattern, the two or more devices mounted to the printed circuit board.

In a second aspect, a system for disinfecting a fluid includes: at least one ultraviolet (UV) light source; a first reflector having a first reflective surface; a second reflector having a first reflective surface; wherein the first and second reflectors are opposite each other and located a predetermined distance from each other, wherein the first reflector emits a first pattern of UV light having a divergence of less than 10 degrees, wherein the second reflect reflector emits a second pattern of UV light having a divergence of less than 10 degrees, the first pattern and second patterns being different; and wherein at least a portion of the first light pattern travels across a volume two or more times the length of the predetermined distance between the first and second reflectors.

In an embodiment of the second aspect, the first reflector has a first focal point and the light source is positioned proximate to the first focal point of the first reflector. In an embodiment of the second aspect, the system further includes a fluid plenum and wherein the reflectors are located within the fluid plenum. In an embodiment of the second aspect, the reflectors provide a functional dose of irradiation to the fluid via the pattern of UV light. In an embodiment of the second aspect, the system further includes one or more absorbing borders are positioned proximal to reflecting surfaces.

In a third aspect, a system for disinfecting a fluid includes: a reflecting device having: at least two reflective internal surfaces spaced a predetermined distance apart, and one or more light sources emitting a pattern of ultraviolet (UV) light having a divergence of less than 10 degrees, wherein at least a portion of the light pattern travels across from a first reflective internal surface to a second internal surface and back again, and wherein fluid passing through the at least two reflective internal surface receives a functional dose of irradiance from exposure to the pattern of UV light.

In an embodiment of the third aspect, one or more absorbing borders are positioned proximate to said reflective internal surfaces. In an embodiment of the third aspect, the reflecting device includes a ring shape, the ring shape having a plurality of internal reflective surfaces that are perpendicular to the direction of the light source. In an embodiment of the third aspect, the system further includes a fluid moving device that passes fluid through the reflecting device. In an embodiment of the third aspect, the fluid moving device includes an axial ceiling fan. In an embodiment of the third aspect, the one or more light sources are positioned proximate to the at least two reflective internal surfaces of the reflecting device and wherein the light sources are directed towards the at least two reflective internal surfaces of the reflecting device. In an embodiment of the third aspect, the one or more light sources are positioned in the center of the reflecting device or along a peripheral edge of the reflecting device.

Further objects, features, and advantages of the disclosure will be apparent from the following detailed description when taken in conjunction with the following drawings.

### BRIEF DESCRIPTION OF THE SEVERAL VIEWS OF THE DRAWINGS

While the appended claims set forth the features of the present techniques with particularity, these techniques, together with their objects and advantages, may be best understood from the following detailed description taken in conjunction with the accompanying drawings of which:

FIG. 1 is a cutaway view of a first embodiment of a reflecting assembly in accordance with principles of the disclosure;

FIG. 2 is a cutaway and expanded view of a second embodiment of a reflecting assembly in accordance with principles of the disclosure;

FIG. 3a is a side view of the second embodiment shown in FIG. 2;

FIG. 3b is a cutaway view of a mounting element in in accordance with principles of the disclosure;

FIG. 4 is a side view of a third embodiment of a reflecting assembly in accordance with principles of the disclosure;

FIG. 5 is a side view of a fourth embodiment of a reflecting assembly in accordance with principles of the disclosure;

FIG. 6 is a partially exploded view of a fifth embodiment of a reflecting assembly in accordance with principles of the disclosure;

FIG. 7 is a partially exploded view of a sixth embodiment of a reflecting assembly in accordance with principles of the disclosure;

FIG. 8 is an exploded view of a seventh embodiment of a reflecting assembly in accordance with principles of the disclosure;

FIG. 9 is a simplified section view of the first embodiment shown in FIG. 1;

FIG. 10a is a simplified section view of an eighth embodiment of a reflecting assembly in accordance with principles of the disclosure;

FIG. 10b shows a chart displaying the behavior of Equation (1) in accordance with principles of the disclosure;

FIG. 10c shows a chart displaying the behavior of Equation (2) in accordance with principles of the disclosure.

FIG. 11a is simplified section view of a ninth embodiment of a reflecting assembly in accordance with principles of the disclosure;

FIG. 11b is a top view of the ninth embodiment shown in FIG. 11a.

FIG. 11c is an exterior side view of a conical reflector of a portion of FIG. 11b.

FIG. 12a is an exterior side view of a tenth embodiment of a reflecting assembly in accordance with principles of the disclosure;

FIG. 12b shows a simplified section view of the tenth embodiment of the reflecting assembly described in FIG. 12a.

FIG. 13 is a top view of an eleventh embodiment of a reflecting assembly in accordance with principles of the disclosure;

FIG. 14 shows an exploded side view of a twelfth embodiment of a reflecting assembly in accordance with principles of the disclosure;

FIG. 15 is a top view of several different configurations of multiple reflector assemblies in accordance with principles of the disclosure;

FIG. 16 is a schematic section view of a first reflecting system in accordance with principles of the disclosure;

FIG. 17a is a side view of a thirteenth embodiment of a reflecting assembly in accordance with principles of the disclosure;

FIG. 17b is an exploded view of the thirteenth embodiment of a reflecting assembly in accordance with principles of the disclosure;

FIG. 18 is a schematic section view of a second reflecting system in accordance with principles of the disclosure;

FIG. 19 is a schematic section view of a third reflecting system in accordance with principles of the disclosure;

FIG. 20 is a schematic section view of a fourth reflecting system in accordance with principles of the disclosure;

FIG. 21 is a simplified side view of the second through fourth reflecting systems in accordance with principles of the disclosure;

FIG. 22 is a schematic section view of a fifth reflecting system in accordance with principles of the disclosure;

FIG. 23a is a side view of a sixth reflecting system in accordance with principles of the disclosure;

FIG. 23b is a top view of the sixth reflecting system shown in FIG. 23a;

FIG. 24a is a side view of a seventh reflecting system in accordance with principles of the disclosure;

FIG. 24b is a top view of the seventh reflecting system shown in FIG. 24a;

FIG. 25a is a side view of an eighth reflecting system in accordance with principles of the disclosure; and

FIG. 25b is a top view of the eighth reflecting system shown in FIG. 25a.

### DETAILED DESCRIPTION

A description of the systems and methods consistent with embodiments of the present disclosure is provided below. While several embodiments are described, it should be understood that the disclosure is not limited to any one embodiment, but instead encompasses numerous alternatives, modifications, and equivalents. In addition, while numerous specific details are set forth in the following description in order to provide a thorough understanding of the embodiments disclosed herein, some embodiments can be practiced without some or all of these details. Moreover, for the purpose of clarity, certain technical material that is known in the related art has not been described in detail in order to avoid unnecessarily obscuring the disclosure.

The particular wording and terminology used to describe a particular embodiment or combinations of embodiments is an example of one combination and should not be considered as limited of the scope of disclosure or possible combinations claimed in the disclosure. Words like “comprise” or “comprising” are used to described how features and steps are combined to illustrate the disclosure but do not preclude the inclusion of additional steps and features or alternate combinations of thereof. Terminology used here has an intended meaning of that familiar to someone who is familiar with the state of the art and in the case that terminology is ambiguous or incorrect, it should not limit the intended meaning.

The disclosure described herein have been conceived and developed with the goals of improving the level of disinfection achieved by a given amount of optical output power while reducing the exposure to room contents below thresholds of concern, reducing costs, and enabling a convenient, safe, and reliable user experience.

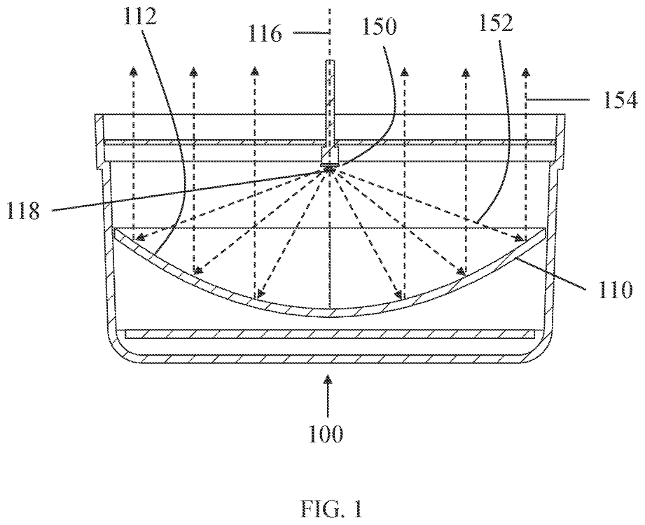

With reference to the drawings, FIG. 1 is side cutaway view of an embodiment of a reflecting assembly, indicated generally at 100. Reflecting assembly 100 includes a reflector 110 and a light source 150. Reflector 110 includes a reflective surface 112. In some embodiments, reflective surface 112 is paraboloidic in shape, as shown.

Reflector 110 further includes at least one focal point 118. As shown in FIG. 1, in some embodiments, focal point 118 is positioned along the central axis 116 of reflective surface 112 and the light source 150 is also positioned along the central axis of the reflector. In some embodiments, the light source 150 is co-located with the geometric focal point 118 of the reflective surface 112. A paraboloid shaped reflector 110 generally has a geometric focal point 118 where incident rays that are parallel to the axis of the paraboloid may all reflect through a single point, the focus. Alternatively, rays emanating from the focal point 118 may reflect off the paraboloid shape and exit parallel to the axis of the paraboloid.

In operation, light source 150 emits at least one beam of direct light 152 that makes contact with reflective surface 112. Once contact is made, reflective surface 112 reflects the light back as at least one beam of reflected light 154. As shown, for each beam of direct light 152, there is a corresponding beam of reflected light 154.

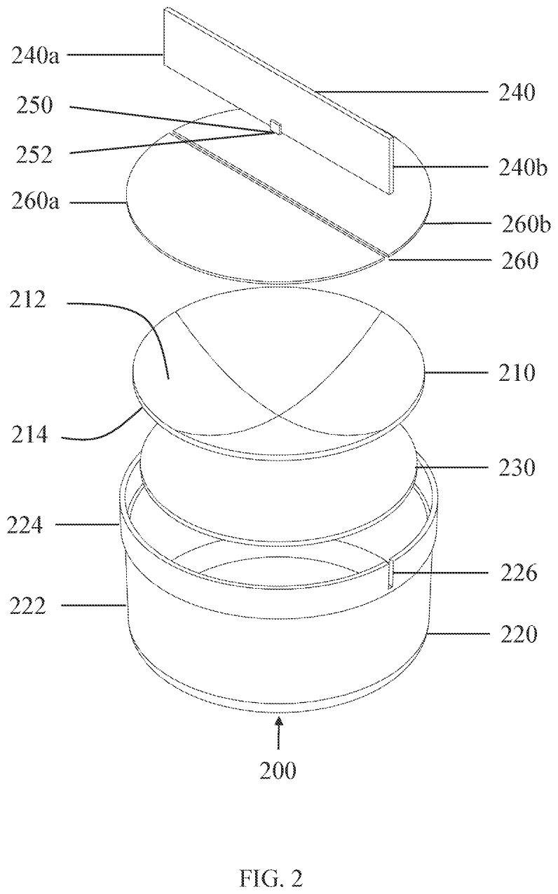

FIG. 2 is a side cutaway and expanded view of an embodiment of a reflecting assembly, indicated generally at 200. Reflecting assembly 200 includes a reflector 210, a housing 220, and a circuit board 230. In some embodiments, reflector 210 includes a generally parabolic or paraboloidic base portion that reflects light in a desired manner 212 and a lip or collar portion 214 that may be used to position and retain the reflector 210.

Housing 220 may include a body portion 222 and a collar portion 224. In some embodiments, collar portion 224 includes one or more notches or slots 226. The one or more notches or slots 226 may be used to align or receive one or more mounting elements 240. As shown, a single mounting element 240 may be received by two slots 226 at mounting element ends 240a and 240b, which as shown are 180 degrees apart.

Also shown in FIG. 2, is a light source 250 mounted to the center of mounting element 240. Any suitable method of mounting light source 250 to mounting element 240 may be used, such as e.g., soldering, adhesive, clamping, welding, or the like. In some embodiments, the light source 250 will be attached to an intermediate structure like a metal-core circuit board (not shown) to make electrical and thermal connection to the light source 250. A window or lens 260 may be provided between mounting element 240 and reflector 210. In some embodiments, lens 260 may be provided as two or more sections, such as shown as 260a and 260b. Lens 260 may serve to keep reflector 210 free from debris such as dust and may serve to isolate the reflector 210 and light source 250 from the surrounding fluid.

Light source 250 may be any suitable lighting element, such as light emitting diodes (LEDs), excimer lamps, mercury lamps, or the like. In some embodiments, LEDs may be used that operate in the ultraviolet (UV), visible, and/or infrared (IR) range. In some embodiments, the range may be selected from approximately 180 nm to approximately 415 nm, and in some embodiments between 180 nm and 280 nm.

In some embodiments, circuit board 230 may include a circuit (not shown) to provide controlled power to the light source 250, a programmable processor or chip (not shown) for executing commands, one or more sensors (not shown) such as temperature sensors and irradiance sensors, as well as a radio for communication electrically connected to the light source 250 through one or more interconnecting devices. For clarity, however, not all of the electrical connections are shown. Circuit board 230 may include electronic circuitry to receive ordinary household current from conductive prongs (not shown) and provide power to illuminate light source 250. Circuit board 230 may include an energy stabilizer such as a full wave rectifier circuit or any other circuit that provides steady voltage to light source 250.

In some embodiments, light source 250 is shown as one or more LEDs 252 (not shown). In such embodiments, circuit board 230 may provide power to one or more LEDs 252 to provide UV and/or visible and/or IR light, although it may be configured to provide power to only UV LEDs 252 or to only visible light LEDs 252 or to only IR light LEDS 252, or to provide variable power to produce combinations of flickering UV and/or visible and/or IR light. In some embodiments, light source 250 may be a low pressure mercury lamp, a high pressure mercury lamp, an amalgam lamp, an excimer lamp, or any other light source that emits wavelengths in the range of interest.

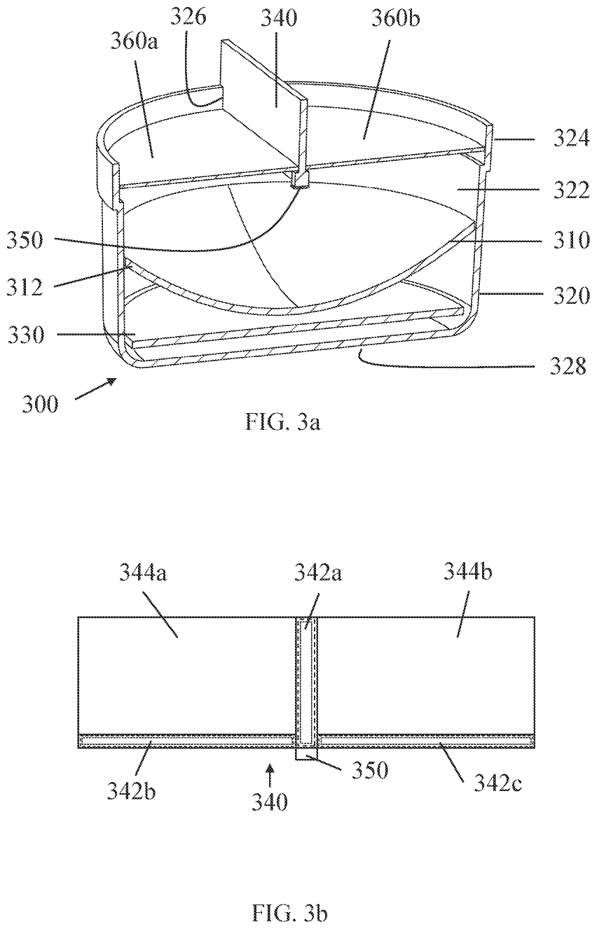

FIG. 3a is an alternative side view of the reflecting assembly shown in FIG. 2. A reflecting assembly, indicated generally at 300, includes a reflector 310, a housing 320, and a circuit board 330. Inner surface 322, collar portion 324 and bottom portion 328 are shown as part of housing 320.

Mounting element 340 is shown as being received by a slot 326 and located proximate to two lens portions 360a and 360b. A light source 350 is shown mounted to the bottom side of mounting element 340.

In some embodiments, mounting element 340 is fabricated from one or more thermally conductive materials such as aluminum, copper, or thermally modified polymer. In such embodiments, mounting element 340 is configured to remove heat from light source 350, thereby functioning as a heat sink. In some embodiments, mounting element 340 is configured to remove at least 50% of the heat generated from light source 350, more preferably configured to remove at least 70% of the heat generated from light source 350, and most preferably configured to remove at least 90% of the heat generated from light source 350.

Still referring to FIG. 3, reflector 310 is shown with its edge 312 in contact with inner surface 322 of housing 320. Reflector 310 may be fabricated from any surface that will specularly reflect incident light rays of the wavelength in use. As used herein, a specular reflection is one where incoming rays of light incident to a surface are redirected away from said surface according to the law of reflection, where the angle between the incoming ray direction vector and the surface is equal to the angle between the surface and the outgoing ray direction. In contrast, a diffuse reflection is one where the reflected light is redirecting into many different directions. Generally, when light strikes a surface, some portion will be absorbed, some portion will be reflected specularly, and some portion will be reflected diffusely. The relative ratio of these components depends on the surface material and finish and is specific to a wavelength. Because reflection is a surface interaction, the composition of the reflecting layer may be selected to achieve desired reflective results.

In practice, an optically transmissive material may be used in conjunction with a reflecting layer such that incident and reflected light passes through the transmissive material in order to protect and establish the surface finish of the reflecting layer and prevent the interaction between the reflecting layer and the environment. In some embodiments, reflector 310 may be produced as a component from aluminum where the outer layer of aluminum is finished and/or coated to give a desired specular reflection. The aluminum may be produced and finished in sheet form and then formed into shape or manufactured to the component form and then finished. Alternately, reflecting and protective layers may be deposited as a thin film on at least a portion of a base component like plastic, ceramic, or metal. As used herein, finishing may include a process to smooth and stabilize the outer surface of the component and may also include a process to apply or induce a protective, transmissive over-coat.

In some embodiments, reflector 310 is formed from sheet metal such as aluminum that is pre-finished in sheet form to produce a specularly reflective surface having a specular reflection of greater than 80%. The metal sheet may be deformed into the desired paraboloid shape using mechanical pressure and the resulting component has enough structural integrity to maintain shape in operation. In some embodiments, reflector 310 is produced from a thin pre-finished sheet of metal and is attached to another component in the desired paraboloid shape such that the mechanical stress on the sheet material is low and the separate component establishes the shape and resists mechanical forces in operation. In some embodiments, reflector is 310 is produced from a thin film of aluminum, silver, gold, or other reflecting material applied to the base component using a metallization process such as photo-vapor-deposition, flame spraying, electroplating, or two-part silvering.

In some embodiments the reflecting layer may be applied to an optically transmissive substrate component such that incident and reflected rays pass through said substrate component in operation. In some embodiments, the reflecting layer is applied to a substrate component such that the incident and reflected rays do not pass through the substrate component and a protective transmissive layer may also be applied on top of the reflecting layer. A protective material such as SiO2 may be deposited over the reflecting layer using photo-vapor-deposition or another method for applying a thin film. In some embodiments the reflective layer may be deposited in a thickness between approximately 0.05 um and 5 um.

FIG. 3b shows an isolated and cutaway view of one embodiment of the mounting element in reflecting assembly 300 indicated generally at 340. In some embodiments, mounting element 340 is fabricated as a thermal module. For example, one or more first portions 342a, 342b, 342c may be constructed as sealed metal sleeves each filled with a mixture of working fluids, and one or more second portion 348a, 348b may be constructed from copper, aluminum, or other thermally conductive material. In such embodiments, the one or more first portions of mounting element 342a, 342b, 342c may function as evaporative heat pipes, thereby transferring heat along each axis to one or more interfaces with the second portions of mounting element 344a, 344b. Thus, it may be advantageous to have first portions of mounting element 342a, 342b, 342c be proximate to and in contact with light source 350 to pull the heat from light source 350, thereby operating as a heat pipe, and have second portions of mounting element 344a, 344b be proximate to and in contact with the heat pipes, so that second portions operate as a heat sinks, transferring heat from light source 350 to the surrounding fluid.

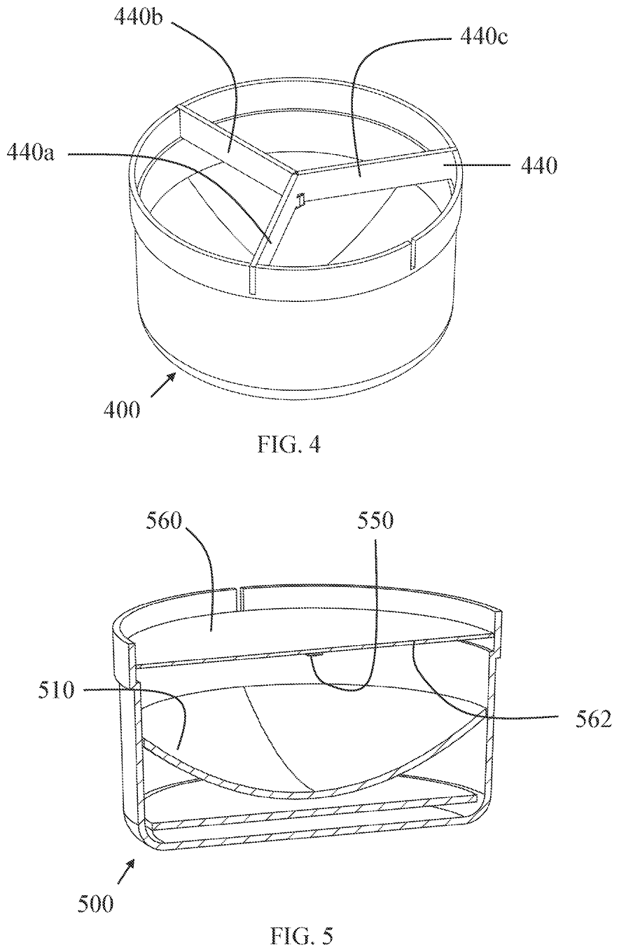

Turning now to FIG. 4, FIG. 4 is a side view of a third embodiment of a reflecting assembly, indicated generally at 400. As shown, reflecting assembly 400 includes a mounting element 440 made up of three arms 440a, 440b, 440c. It should be appreciated that while three arms 440a, 440b, 440c are shown, any suitable number of arms may be used. In some embodiments, mounting element 440 is selected to occlude a small amount of reflected light from the light source from exiting the reflecting assembly 400. In some embodiments, preferably less than 15% of the reflected light will be occluded, and more preferably less than 5% will be occluded.

FIG. 5 is a side view of a fourth embodiment of a reflecting assembly, indicated generally at 500. Reflecting assembly 500 includes a window or lens 560, a reflector 510, and a light source 550. As shown, a light source 550 is mounted to lens 560. It should be appreciated that, lens 560 thereby functions as a mounting element, heat sink, and electrical interconnection, in addition to preventing dust or debris from getting into reflecting assembly 500. In some embodiments, electrical connection between the light source 550 and the power circuit (not shown) may be one or more electrically and thermally conductive elements 562 that are bonded or applied to lens 560. In some embodiments, said conductive elements may be a thin film that is deposited onto the lens 560 directly. In some embodiments, said conductive elements are prefabricated and bonded to the lens using adhesive, for example a printed circuit that is applied to the lens using pressure sensitive adhesive.

In the fourth embodiment of a reflection assembly described in FIG. 5, the light source 550 may be connected to the conductive elements 562 using solder, conductive adhesive, welding, or the like.

In some embodiments, window or lens 560 is least partially transparent or translucent. For example, lens 560 may be fabricated from silicon dioxide (SiO2) or sapphire (Al2O3) that is transmissive to UV light. In some embodiments, lens 560 is transmissive to one or more of UV-A, UV-B, and UV-C light. As defined herein, UV-A includes wavelengths from 315 nm-400 nm, UV-B includes wavelengths from 280 nm-315 nm, and UV-C includes wavelengths from 100 nm-280 nm.

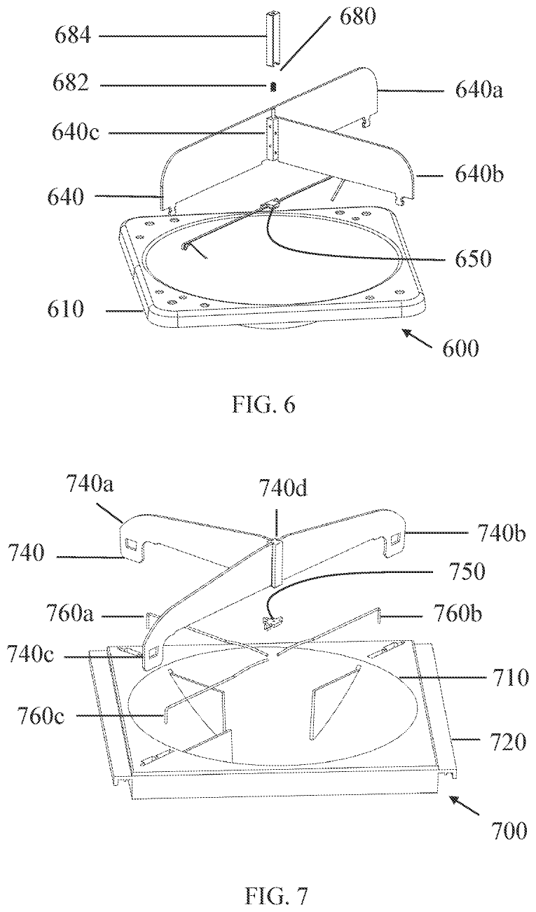

FIG. 6 is a partially exploded view of a fifth embodiment of a reflecting assembly, indicated generally at 600. Reflecting assembly 600 includes a reflector 610, a mounting element 640 made up of two arms 640a, 640b, and a mounting hub 640c, and a light source assembly 650, such as an LED attached to a printed circuit board (PCB). As shown, reflector 610 and mounting element 640 are formed from sheet metal. In some embodiments, reflecting assembly 600 also includes a tension element 680, such as a spring 682 combined with a load transferring bracket 684 to ensure good thermal connection between the light source 650 and mounting element 640.

It should be appreciated that a low resistance to heat transfer across a component interface in a thermal path, e.g., such as that between light source 650 and mounting element 640, enables effective temperature control of the light source 650. In the case of two separate components that are not welded or otherwise bonded together, a low heat transfer resistance across an interface may be achieved by ensuring the mating surfaces have a similar shape, thereby limiting the size of air gaps between contact points. When a compressive force is applied across the interface, the mating surfaces will contact each other at a plurality of discrete contacts points across the surface and in some cases, the resistance to heat transfer across the interface is known to increase as the pressure across the interface is increased. Additionally, a compressive force will push two components together, thereby fixing the relative positions in the face of operational forces. Using this construct, in some embodiments, light source assembly 650 and the mounting element 640 are in contact via compression or a compressive force to promote good thermal performance and set relative position. Such compressive force may be applied via tension element 680, as described below. In some cases, it is also advantageous to include a compliant, heat conducting material in between the mating surfaces of such as joint. A joint includes two interfacing exterior surfaces of two or more components and any interstitial material such as a liquid, solid, or gas.

In operation, tension element 680 may provide a compressive force between the light source assembly 650 and the mounting element 640. For example, tension element 680 may be in contact with mounting hub 640c at one end, and one end attached to the light source assembly 650. Because the light source assembly 650 is also in contact with mounting element 640, which may be configured to operate as a heat sink (as described in other embodiments throughout this specification), the tension element 680 aids in removing heat from the light source assembly 650.

It should be appreciated that while tension element 680 is shown as a coil spring 682 and load transferring bracket 684, any mechanical element or combination of one or more mechanical elements that can apply a load across the joint interface and have a stiffness of usable magnitude across an operating displacement of usable magnitude will achieve the goal of applying a compressive force across the joint. For example, a wire spring, torsion spring, wave spring, or the structural deformation modes of mounting element 640, reflector 610, or light source assembly 650.

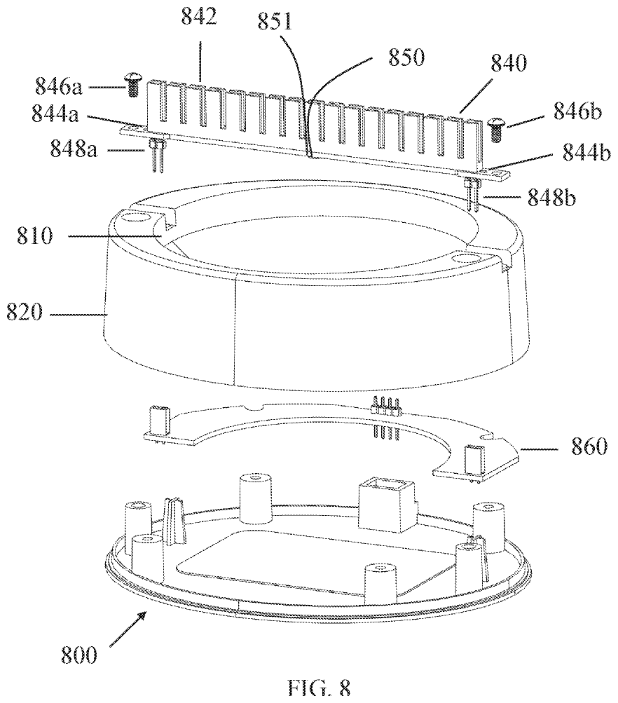

FIG. 7 is a partially exploded view of a sixth embodiment of a reflecting assembly, indicated generally at 700. Reflecting assembly 700 includes a reflector 710, a housing 720, a mounting element 740 made up of three arms 740a, 740b, 740c, a mounting hub 740d, a light source assembly 750, and wire springs 760a, 760b, 760c. As shown, mounting element 740 is formed from metal sections that are bonded together. As shown, housing 720 is made from molded plastic with features that receive, retain, and positions the mounting element 740. Reflector 710 may have a reflective surface as described in FIG. 3. Wire springs 760a, 760b, and 760c may be located and retained into the housing 720.

In operation, the wire springs 760a, 760b, and 760c are compressed from their rest state when the mounting element 740 and light source assembly 750 are installed in the housing 720. This compression generates a force between the light source 750 and the mounting element 740, which results in good thermal connection at the interface as well as maintaining relative position, and this force is sustained by the structural connection between the mounting element 740 and housing 720. Additionally, one or more of the wire springs 760a, 760b, and 760c may make electrical connection with the light source assembly 750 in order to power or communicate with the light source assembly 750.

FIG. 8 is an exploded view of a seventh embodiment of a reflecting assembly, indicated generally at 800. Reflecting assembly 800 includes a reflector 810, a housing 820, a mounting element 840, a light source 850, and a PCB 860. In some embodiments mounting element 840 is made from a metal-core PCB, to which the light source 850 is affixed, e.g., via soldering. Metal-core PCBs are generally constructed from layers of conducting circuit elements, thin dielectric insulators, and metal such as aluminum or copper. This results in a printed circuit board that has a lower resistance to heat transfer than a circuit board constructed from ceramic and polymer composites while maintaining the circuit interconnect functionality and manufacturing benefits.

A heat sink 842 is also shown attached to the mounting element 840. The mounting element 840, heat sink 842, and light source 850, may be positioned and attached to housing 820 through one or more structural connections, like e.g., pin joints 844a, 844b and clamping screws 846a, 846b. The mounting element 840 may have circuit traces that connect the light source 850 to other circuit signals and components. For example, electrical connectors 848a and 848b that can interface with connectors attached to PCB 860. Also, the mounting element 840 may include other circuit components such as an irradiance sensor or resistor (not shown).

In operation, heat generated by the light source 850 may be transferred to the mounting element 840 through solder joints 851 that may also provide electrical connection to the light source 850 and other circuit elements. Heat then travels through the mounting element 840 where it is transferred to heat sink 842. Heat is then transferred from the heat sink 842 to the surrounding fluid, e.g., air.

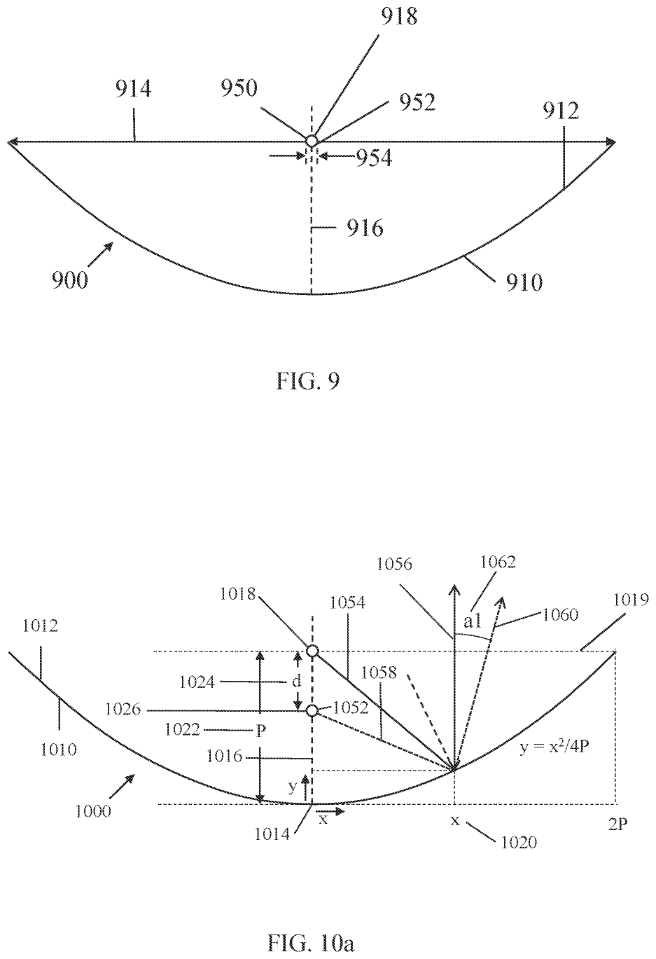

FIG. 9 is a simplified section view of the first embodiment shown in FIG. 1, indicated generally at 900, with dimensions provided. In order for the reflector assembly 900 to function as desired, reflector 910 may be of sufficient size compared to the light source 950. As used herein, the light-emitting area 952 of a light source 950 is the smallest area that passes all of the light rays emanating from the light source 950. The characteristic dimension 954 of the light emitting area 952 is the longest dimension across said area. For example, if the area is circular in nature, the characteristic dimension is the diameter of the circle. If the area is square or rectangular in nature, the characteristic dimensions is the length between two opposing corners. A paraboloid reflector will have a parabolic cross section 912 that receives light from light source 950. The characteristic dimension 914 of reflector 910 is the chord length that is perpendicular to the parabola axis 916 and that passes through focal point 918. There is no minimum value of the light source characteristic dimension 954 in relation to the characteristic dimensions of the reflector 914. However, the characteristic dimensions of the light source 954 may be less than 8% of the characteristic dimension of the reflector 914, preferably less than 4%, and more preferably less than 2%.

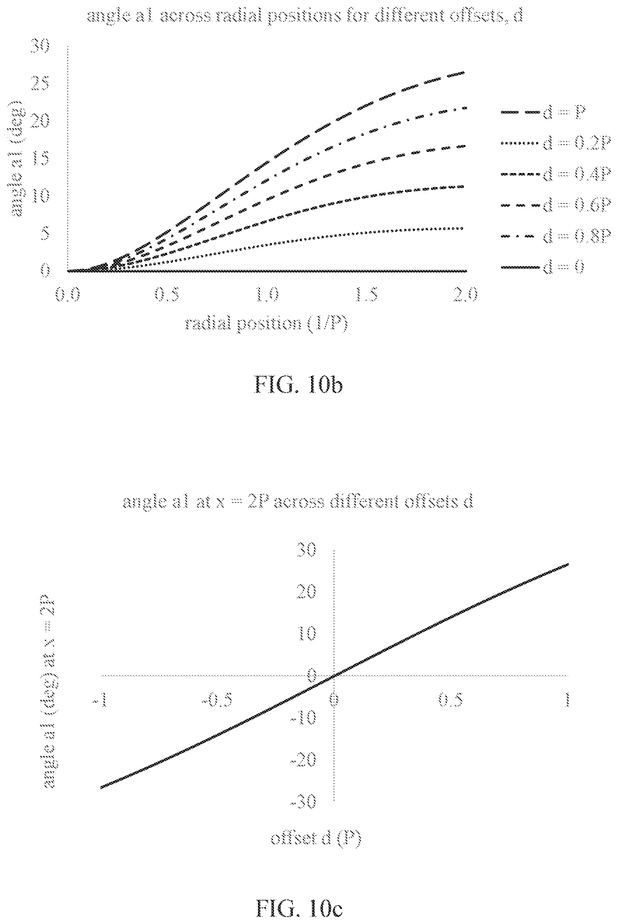

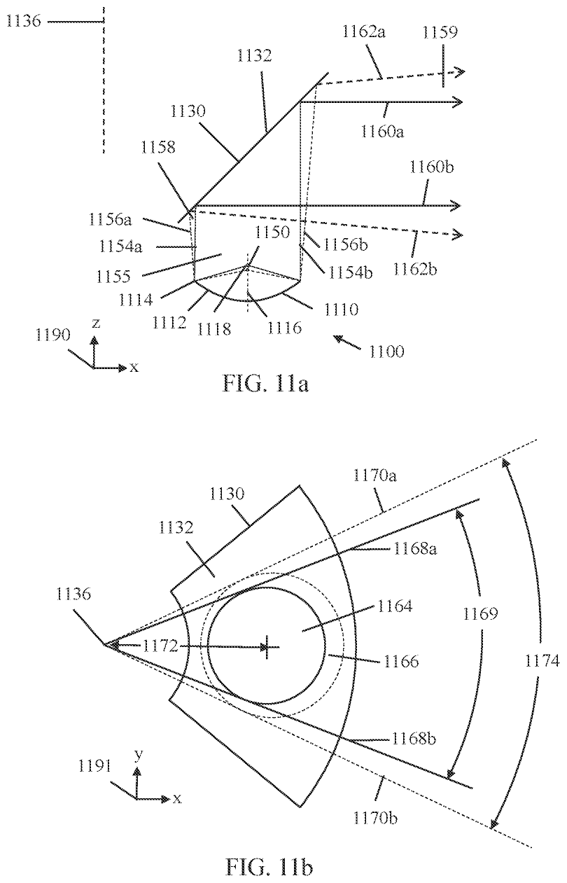

FIG. 10a is a simplified section view of an eighth embodiment of a reflecting assembly, indicated generally at 1000. The reflecting assembly 1000 includes a reflector 1010 with a parabolic cross section 1012 and geometric focal point 1018 that follows the equation y=x²/4P where y is the vertical distance from the parabola vertex 1014, x is the radial distance 1020 from the axis 1016, and P is the vertical distance 1022 between the vertex 1014 and the focal point 1018. A light ray 1054 from the focal point 1018 will reflect off the reflector 1010 in a direction 1056 parallel with the parabola axis 1016. If the light source center 1052 is positioned along the axis 1016 of the parabola at a distance d 1024 below focal point 1018, a light ray 1058 will reflect off the reflector and exit at a direction 1060 which has an angle a1 1062 to ray 1056.

The rays in FIG. 10a are drawn at an arbitrary radial position x 1020. Because the reflector cross section 1012 is a parabola, reflected ray 1056 will be parallel to the axis 1016 for all radial positions of the parabola. At a radial position 1020 equal to two times the focal distance P 1022, the line 1019 between the reflector cross section 1012 and the focal point 1018 is perpendicular to the axis 1016. Trigonometry may be used to determine the relationship between radial position x 1020, distance d 1024, focal length P 1022 and the diverging angle a1 1062 by an Equation (1):

$${a}_{1} = {tan}^{- 1} ( \frac{P}{x} - \frac{x}{4 P} + \frac{d}{x} ) - {tan}^{- 1} ( \frac{P}{x} - \frac{x}{4 P} ) \qquad Equation ( 1 )$$

Note that while in FIG. 10a the light source center 1052 is drawn below the focal point 1018, Equation (1) may also be accurate for cases in which the light source center 1052 is positioned above the focal point. Also, while the parabola is drawn up to a radial position of two times the focal length, Equation (1) may be accurate and relevant for greater radial distances as well.

FIG. 10b shows a chart displaying the behavior of Equation (1) for six different values of offset distance d 1024. Divergence angle a1 1062 is plotted on the vertical axis and radial position x 1020 is plotted on the horizontal axis. In all cases, the divergence angle is zero at a radial position of zero. In the case that offset d 1024 is zero, the divergence angle 1062 is zero across all radial positions. In the cases where there is a non-zero offset, the divergence angle 1062 will increase from zero as radial position 1020 is increased. For greater offset values, the divergence angle will increase to a greater value. Many light rays will exit light source center 1052 at the same time, and each will reflect according to the light source offset distance 1024 and the radial position 1020 at which that specific light ray will reflect off reflector section 1012. Accordingly, the group of reflected rays will have different directions ranging from parallel with the axis 1016 to the divergence angle 1062 corresponding to the largest radial position 1020. In addition to the reflector 1010 geometry, the distribution of power across emission angles in the light source will affect how the optical power is distributed across these direction. In some cases, it is convenient to characterize the divergence of an aggregate light source as the divergence angle 1062 of the light ray at some reference radial position. For example, the divergence 1062 of the light rays contacting the reflector section 1012 at a radial position 1020 equal to two times the focal length P 1022.

FIG. 10c shows a chart displaying the diverging angle 1062 at radial position 1020 equal to two times the focal length P 1022 for different values of offset d 1024. Diverging angle 1062 is plotted on the vertical axis, and offset value 1024 is plotted on the horizontal axis. Equation (1) may be simplified by evaluating it at a radial position x 1020 equal to two times the focal length P 1022. This simplified form is shown as Equation (2):

$${a}_{1} = {tan}^{- 1} ( \frac{d}{2 P} ) \qquad Equation ( 2 )$$

Equation (2) shows that the greatest diverging angle 1062 will increase as offset d 1024 is increased, and that the diverging angle 1062 will reverse in the case that negative offset values 1024 are used, which correspond to cases where light source center 1052 is above the focal position 1018, and the reflected ray 1060 is pointed towards axis 1016 instead of away from it. The case with a negative divergence angle is sometimes referred to as converging.

Therefore, the aggregate divergence of a set of light rays from reflector assembly 1000 may be programmably controlled by changing the offset distance 1024 between the focal point of the paraboloid reflector 1018 and the light source center 1052.

FIG. 11a is simplified section view of a ninth embodiment of a reflecting assembly, indicated generally at 1100. The reflecting assembly 1100 includes a light source 1150, a first reflector 1110, and a second reflector 1130. In some embodiments, reflector 1130 has a section profile 1132 that is axially symmetric about reflector axis 1136. In some embodiments reflector 1110 is a paraboloid, has a section profile 1112, focal point 1118, and is axially symmetric about reflector axis 1116. As shown, light source 1150 is positioned along the reflector axis 1116. In some embodiments, reflector axis 1116 is parallel to reflector axis 1136 and reflector 1110 is positioned such that the exiting rays will interact with reflector 1130.

As described in previous embodiments, light rays will exit light source 1150, reflect off reflecting surface 1112, and exit reflector 1110 with some characteristic divergence pattern dependent on the position of the light source 1150 relative to the focal point 1118 and other factors. The divergence may be fixed or adjustable as described above. Illustrated in FIG. 11a are rays 1154a, 1154b that exit the reflector 1110 parallel to reflector axis 1116 at the edge 1114 of reflecting surface 1112.

Also shown are rays 1156a, 1156b that exit the reflector 1110 with divergence angle 1158. Many different light rays will simultaneously exit light source 1150 and reflect off reflecting surface 1112, and the aggregate nature of these light rays may be referred to as the light pattern as indicated at 1155. In some embodiments, the axially symmetric nature of a paraboloid shape will result in light pattern 1155 being generally axially symmetric in shape. At one extreme, light pattern 1155 may be parallel with zero divergence. In other configurations, light pattern 1155 will consist of a variety of light rays with directions ranging from parallel with the axis 1116 of the reflector to some diverging angle 1158 from reflector axis 1116. It may be convenient to describe light rays at the edge 1114 of the reflector surface 1112 at some maximum divergence angle 1158 to characterize the light pattern because said light rays will bound the position and direction of all possible light rays in pattern 1155.

Rays 1154a 1154b as well as all other rays parallel with axis 1116 may reflect off reflector surface 1132 at a consistent angle to reflector axis 1136 according to the law of reflection as indicated at 1160a 1160b. Rays 1156a 1156b may reflect off reflector surface 1132 according to the law of reflection as indicated at 1162a 1162b. Note that the relative angle 1159 between diverging rays 1162a, 1162b and parallel rays 1160a, 1160b is the same as divergence angle 1158 when exiting reflector 1110. It may be appreciated that reflector 1130 is then able to redirect light pattern 1155 without changing the divergent properties of said pattern within the section plane xz 1190 shown in FIG. 11a.

FIG. 11b is a top view of the ninth embodiment shown in FIG. 11a, indicated generally at 1100. Reflector 1130 may have a conical shape, being axially symmetric about axis 1136. The area of the reflector surface 1132 that will contain the rays exiting reflector 1110 parallel to axis 1116 is indicated at 1164. Similarly, the area of reflector surface 1132 that will contain divergent rays such as 1156a 1156b is indicated at 1166. Any ray exiting reflector 1110 will strike reflecting surface 1132 and exit according to the law of reflection.

For rays that are parallel to reflector axis 1136, the outgoing direction in the view shown may be found by drawing a line from axis 1136 to the point on the reflecting surface 1132 where the ray is incident. Lines 1168a and 1168b represent the outgoing direction of the outermost rays in area 1164. Other rays exiting reflector 1110 parallel to axis 1136, and striking reflector 1132 within area 1164 will have outgoing directions between lines 1168a and 1168b, and the aggregate light pattern can then be described to have divergence angle 1169. The radial distance between axis 1136 and the center of areas 1166 is indicated at 1172.

The outgoing direction of rays reflecting off reflector surface 1132 that exit reflector 1110 with some divergence such as 1162a and 1162b may also generally be found by drawing a line between reflector axis 1136 and the point of reflection on reflector surface 1132 as represented by lines 1170a and 1170b. However, compared to the case of rays parallel to axis 1116, this approach is more of an approximation. The divergence angle in between lines 1170a and 1170b is indicated at 1174. It should be appreciated that reflector 1130 is able to change the divergence of light pattern 1155 selectively in the direction of the plane xz 1191 shown in FIG. 11b.

As shown in FIG. 11a and FIG. 11b, reflector 1130 may transform a pattern of light rays 1155 exiting reflector 1110 with axial symmetry by redirecting it and selectively modifying or propagating the divergence in two orthogonal directions.

FIG. 11c is an exterior side view of a conical reflector indicated generally at 1130. The reflector 1130 includes a conical surface 1132, and four discrete sub-sections 1166a, 1166b, 1166c, and 1166d. Each of the discrete sub-sections 1166a, 1166b, 1166c, and 1166d correspond to a different radial position 1172. Generally, the angle 1174 corresponding to each subsection 1166a, 1166b, 1166c, and 1166d will change based on the radial position 1172 of each.

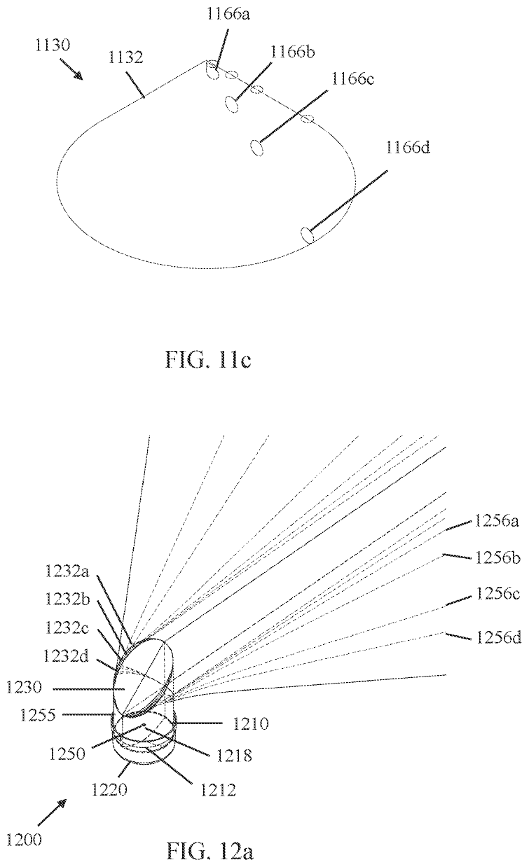

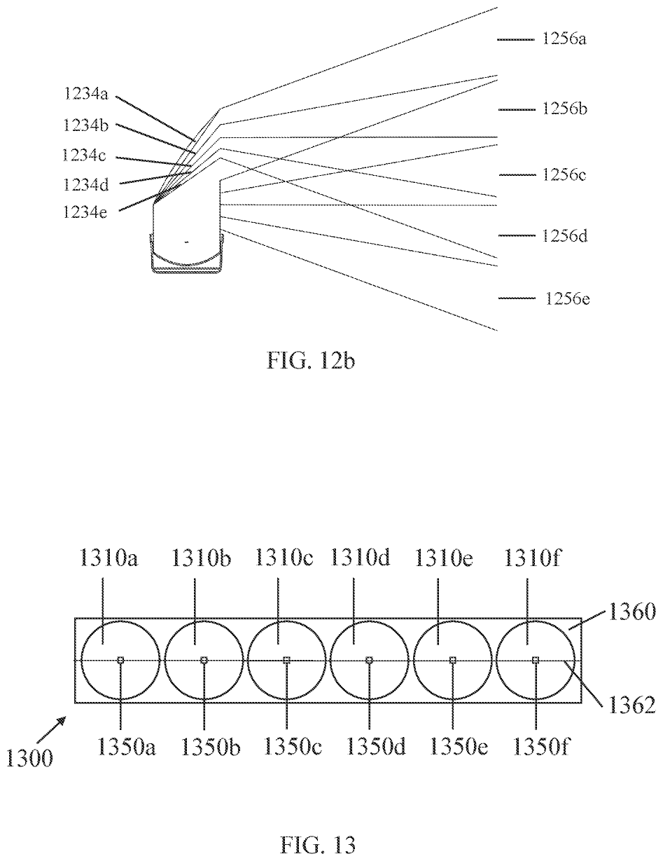

2| FIG. 12a is an exterior side view of a tenth embodiment of a reflecting assembly, indicated generally at 1200. Reflecting assembly 1200 includes a light source 1250, a first reflector 1210 of paraboloid shape with reflecting surface 1212, a housing 1220, and a second reflector 1230 that is configured to reflect a pattern of light rays 1255 exiting reflector surface 1212. As described in FIG. 11a, FIG. 11b and FIG. 11c, the shape and position relative to reflector surface 1212 of reflector surface 1232 may be selected to achieve a desired output pattern of light rays from said surface.

FIG. 12a shows four different conical shapes corresponding to the four conical subsections described in FIG. 11c superimposed for illustrative purposes indicated at 1232a, 1232b, 1232c, and 1232d. Also shown are the divergent light patterns reflecting off each respective conical shape 1256a, 1256b, 1256c, and 1256d.

FIG. 12b shows a simplified section view of the tenth embodiment of the reflecting assembly described in FIG. 12a. Reflector 1230 is drawn with five discrete conical angles 1234a, 1234b, 1234c, 1234d, and 1234e and the reflected output pattern is shown in five corresponding directions 1256a, 1256b, 1256c, 1256d, and 1256e.

In some embodiments, the shape of reflecting surface 1230 may deviate from the conical shape described for the purposes of manufacturing convenience or for functional reasons. For example, a cylindrical shape or other similar shapes may be used.

In some embodiments, reflector 1230 may be adjustable in nature, such as through means of adjustable mounting, deformation, etc. This configuration may be done at the time of construction, deployment, or during use and may or may not be carried out simultaneously with adjustments to the divergence of light pattern 1255 by means of adjusting the position of light source 1250 relative to the focal position 1218 of reflecting surface 1212.

In some embodiments, the shape of reflector 1230 and/or the position of light source 1250 relative to the focal point of reflecting surface 1212 may be continuously adjusted using a motorized system that can change the shape and position of the reflectors on demand.

FIG. 13 is a top view of an eleventh embodiment of a reflecting assembly, indicated generally at 1300. Reflecting assembly 1300 includes a plurality of reflectors 1310a, 1310b, 1310c, 1310d, 1310e, 1310f and a plurality of respective light sources 1350a, 1350b, 1350c, 1350d, 1350e, 1350f. In some embodiments, a single lens 1360 may be mated to multiple reflectors 1310a, 1310b, 1310c, 1310d, 1310e, 1310f and light sources 1350a, 1350b, 1350c, 1350d, 1350e, 1350f. In some embodiments, a single circuit of conducting elements 1362 that are in electrical connection with one or more power sources may be used to power one or more light sources 1350a, 1350b, 1350c, 1350d, 1350e, 1350f. In some embodiments lens 1360 may be at least partially optically transmissive and continuous across the area of the reflectors 1310a, 1310b, 1310c, 1310d, 1310e, 1310f.

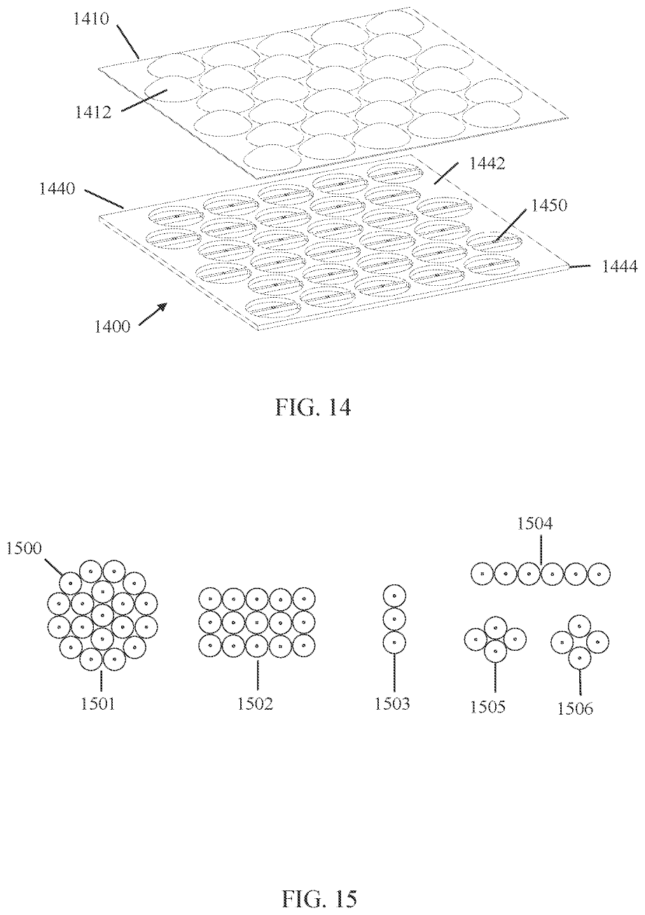

FIG. 14 shows an exploded side view of a twelfth embodiment of a reflecting assembly, indicated generally at 1400. Reflecting assembly 1400 includes a mounting element 1440 to which one or more light sources 1450 are attached, and further includes a reflecting sheet 1410 with one or more reflecting surfaces 1412 that interfaces with mounting element 1440 and reflects the output of light sources 1450 according to the principles described above. Mounting element 1440 may be attached to reflecting sheet 1410 through adhesive, solder, welding, one or more clamping interfaces, or the like. In some embodiments, mounting element 1440 is selectively perforated to allow reflected light to pass while still maintaining electrical and thermal connection with the light source 1450. In some embodiments, mounting element 1440 is manufactured as a printed circuit board with circuit layers and interconnections 1442 integrated into its construction. In some embodiments, mounting element 1440 is manufactured as a metal core PCB with a highly thermally conductive substrate layer 1444 separating and supporting one or more circuit layers 1442. In some embodiments, a transmissive window (not shown) may be included in reflecting assembly 1400 that protects reflecting surfaces and/or light sources from dust, debris, and/or surrounding fluid.

FIG. 15 is a top view of several different configurations of multiple reflector assemblies 1501, 1502, 1503, 1504, 1505, and 1506. In each configuration, a plurality of reflector assemblies 1500 are positioned adjacent to each other with the axes of their reflected patterns parallel to each other. In operation, one or more reflecting assemblies 1500 may be operated simultaneously. The resulting aggregate pattern will then have characteristics determined by the individual reflected patterns.

In many lighting applications the irradiance delivered to a surface is the functional metric being optimized. For instance, in illumination lighting and surface processes such as curing, the planar irradiance at a surface is a metric of concern. In contrast, in some applications where a bulk fluid is subjected to light for functional purposes such as the disinfection of air and water, the spherical irradiance across the volume being treated is a metric of concern. Spherical irradiance is a property of optical systems that exists at a given point in space. It has the same units as planar irradiance, radiometric or optical power per square distance unit, but represents power arriving at a point from all directions uniformly.

For example, in fluid disinfection with UV-C irradiation such as air disinfection, the functional effect may be quantified according to the spherical irradiance aggregated across the volume being treated by multiplying the average irradiance by the room volume or integrating a spatially variable irradiance field across the volume. In some cases, it is desirable to maximize this aggregate irradiance value for a given optical power introduced into a volume. Generally, the cost to deploy and operate an optical system scales with the amount of power being used. It may be shown that the aggregate irradiance achieved in an optical system is directly proportional to the path length that each light ray travels before being attenuated by being absorbed or exiting the volume. Accordingly, it is often desirable to deploy optical systems that maximize the path length of an optical source within a volume being treated.

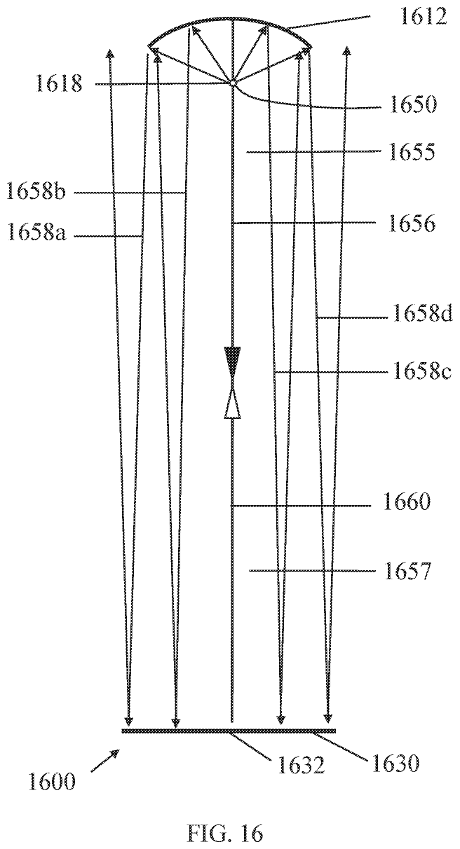

FIG. 16 is a schematic section view of a first reflecting system, indicated generally at 1600. Reflecting system 1600 includes a paraboloid reflecting surface 1612, a light source 1650, a reflected light pattern 1655 exiting reflecting surface 1612, and a secondary reflector 1630 with reflecting surface 1632. In some embodiments, the divergence of reflected light pattern 1655 is minimized such that the cross sectional area of the light pattern 1655 does not increase excessively along the path length 1656, thereby limiting the size of reflecting surface 1632 needed to capture some portion of light pattern 1655. Some rays of pattern 1655 are shown by 1658a, 1658b, 1658c, and 1658d. In operation, these rays 1658a, 1658b, 1658c, and 1658d and the rest of light pattern 1655 are reflected off of reflecting surface 1632 and the reflected pattern 1657 will have a path 1660 opposite of the incoming light pattern. In some embodiments, reflecting surface 1632 is planar in shape. In other embodiments, reflecting surface 1632 has a shape that will transform the light pattern 1655 in some fashion such as increasing or decreasing divergence. A portion of light pattern 1657 may be incident on reflecting surface 1612 and reflect back towards the focal point 1618 of surface 1612. A portion of pattern 1657 may bypass reflecting surface 1612. It should be appreciated that the spherical irradiance within the volume between reflecting surface 1612 and reflecting surface 1630 may be significantly increased by the inclusion of a highly reflective surface at reflector 1630. Such highly reflective surface may have a specular reflection of greater than approximately 70%.

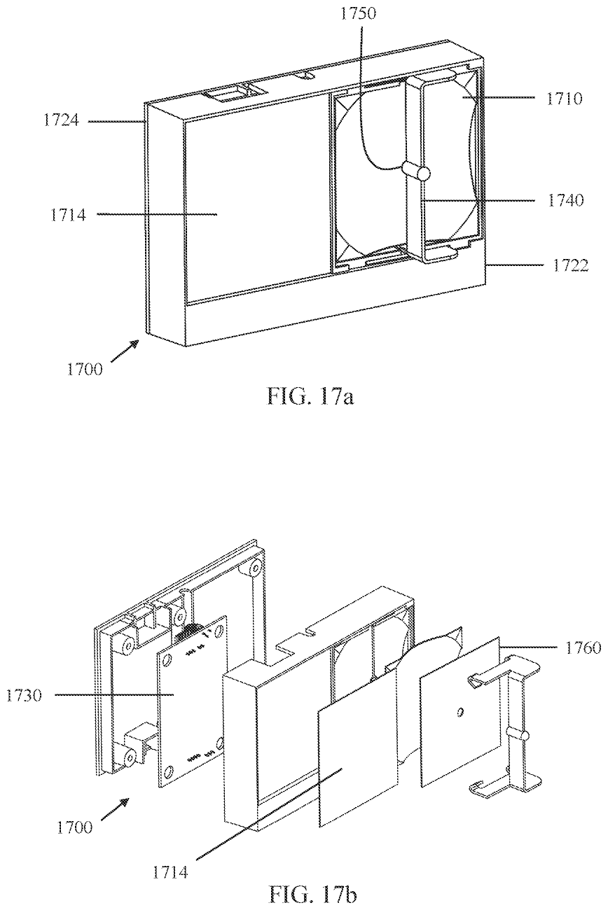

FIG. 17a is a side view of a thirteenth embodiment of a reflecting assembly, indicated generally at 1700. The reflecting assembly 1700 includes a first reflector 1710, a front housing 1722, a back housing 1724, a mounting element 1740, a light source assembly 1750, and a second reflector 1714. In some embodiments second reflector 1714 is planar in shape.

FIG. 17b is an exploded view of the thirteenth embodiment of a reflecting assembly 1700. Also shown are a circuit board 1730 and a lens 1760. In operation, the reflecting assembly 1700 may be used in conjunction with other reflecting assemblies as described below. Light exits light source 1750 and reflects off reflector 1712, exiting reflecting assembly 1700. In some embodiments one or more separate reflecting surfaces (not shown) will redirect the light reflected off reflector 1712 back towards reflecting assembly 1700 where a portion of the light will strike reflector 1714 and reflect back away from reflecting assembly 1700.

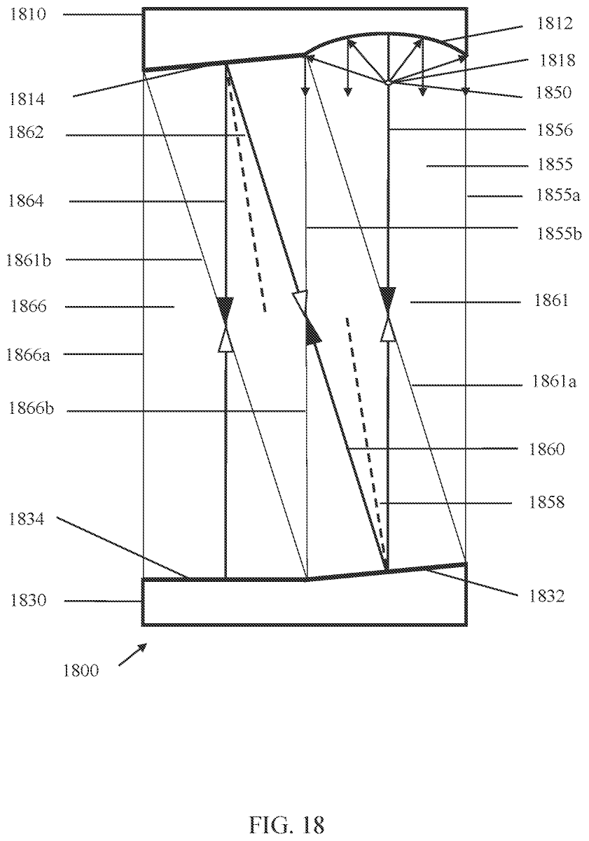

FIG. 18 is a schematic section view of a second reflecting system, indicated generally at 1800. Reflecting system 1800 includes reflecting assembly 1810 and reflecting assembly 1830. Reflecting assembly 1810 includes a paraboloid first reflecting surface 1812, a light source 1850, and a second reflecting surface 1814. Reflecting assembly 1830 includes a first reflecting surface 1832, and a second reflecting surface 1834.

In operation, a light pattern 1855 bounded by rays 1855a and 1855b exits reflector 1812 along path direction 1856 directed towards reflecting surface 1832. Reflecting surface 1832 is positioned at an angle to the central axis of light pattern 1855, indicated at 1858. The light pattern 1855 then reflects off reflecting surface 1832 with an outgoing direction 1860. This second reflected light pattern is shown as bounding rays 1861a and 1861b. Reflecting assembly 1810 includes a reflector 1814 that is positioned along the path 1860 and positioned at angle 1862 relative to the direction of light path 1860. The light pattern reflecting off of reflector 1814, has a direction 1864 and is indicated by bounding rays 1866a and 1866b.

Reflecting surface 1834 is positioned along and perpendicular to path 1864 and light pattern 1866 will reflect off reflecting surface 1834, reversing direction. Because the light pattern reflected off of reflecting surface 1834 travels in the opposite direction of 1864, it will reflect off reflectors 1814 and 1832 in a reverse fashion to the forward path until it arrives back at reflecting surface 1812 and reflects back towards light source 1850.

In FIG. 18 the reflectors 1812, 1814, 1832, 1834 are shown integrated as part of reflecting assemblies 1810 and 1830, but in some embodiments the reflecting assembly and reflectors may be separate components. In some embodiments, the end reflector 1834 may not be included in the system such that the light pattern does not travel the reverse path. In some embodiments, the successive light patterns 1855, 1861, and 1866 are drawn as completely parallel to the axis of travel. However, as discussed previously, light pattern 1855 may exit reflector 1812 with non-zero divergence and the successive reflected light patterns 1861, and 1866 may maintain this divergence. Some of the light may be lost at each reflection interface to absorption at the reflecting surface, to diffuse reflections, and to a portion of the light not being incident on the reflecting surfaces.

It may be desirable to configure a system to maximize the amount of light that passes to each successive reflection. In FIG. 18 the second 1832, third 1814, and fourth 1834 reflecting surfaces are described as planar in nature as is desired in some embodiments. In some embodiments, these reflecting surfaces may be non-planar in nature and have additional effect on the reflected light such as increasing or decreasing divergence.

It should be appreciated that the light exiting reflector 1812 in reflecting system 1800 may traverse the volume between reflecting assembly 1810 and 1830 up to six times, thereby maximizing the spherical irradiance achieved by light pattern 1855. A portion of the light pattern 1855 may travel along path 1856, 1860, and 1864 two times each, once on the path towards end reflector 1834, and once on the return path back to reflector 1812. As used herein the term long-path may be used to describe an optical system where a portion of the light travels at least two times the bounding dimension of the volume within which it operates.

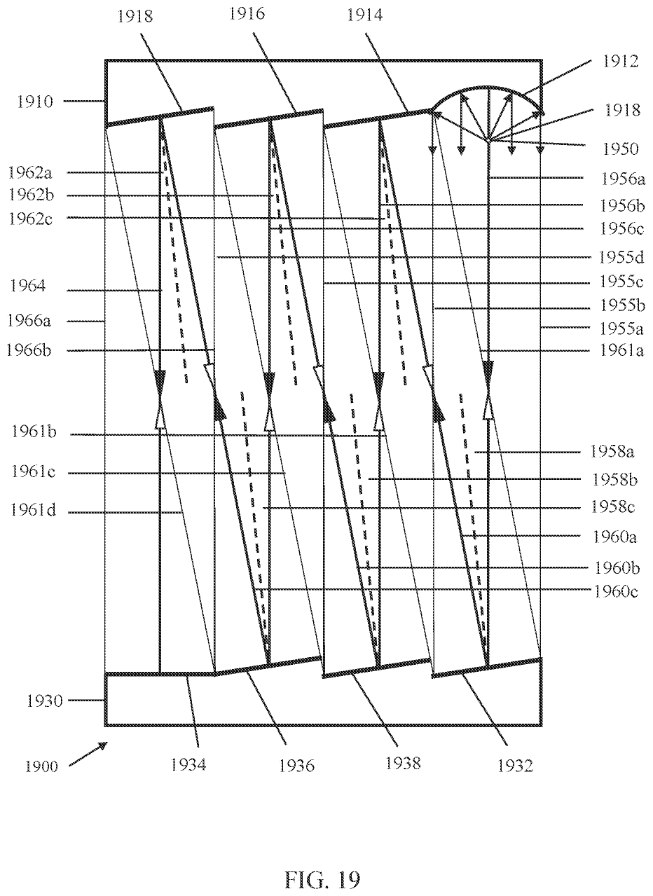

FIG. 19 is a schematic section view of a third embodiment of a reflecting system, similar to that described in FIG. 18 and indicated generally at 1900. Reflecting system 1900 includes a first reflecting assembly 1910 and a second reflecting assembly 1930. Reflecting assembly 1910 includes a paraboloid reflecting surface 1912, a light source 1950 located at the focal point 1918 of reflecting surface 1912, a second reflecting surface 1914, a third reflecting surface 1916, and a fourth reflecting surface 1918. Reflecting assembly 1930 includes a first reflecting surface 1932, a second reflecting surface 1938, a third reflecting surface 1936, and a fourth reflecting surface 1934.

In operation, a light pattern exits reflecting surface 1912 as indicated by bounding rays 1955a and 1955b and travels along path direction 1956a towards reflecting surface 1932. Reflecting surface 1932 is positioned at angle 1958a relative to path direction 1956a and reflects the light. The light pattern exiting reflecting surface 1932 travels along path direction 1960a towards reflecting surface 1914 and is indicated by bounding rays 1961a and 1961b. The light pattern indicated by 1961a and 1961b will reflect off reflecting surface 1914 which is angled at angle 1962c relative to path direction 1960a. The light pattern exiting reflecting surface 1914 travels along path direction 1956b towards reflecting surface 1938 and is indicated by bounding rays 1955b and 1955c. The light pattern indicated by 1955b and 1955c will reflect off reflecting surface 1938 which is angled at angle 1958b relative to path direction 1956b. The light pattern exiting reflecting surface 1938 travels along path direction 1960b towards reflecting surface 1916 and is indicated by bounding rays 1961b and 1961c. The light pattern indicated by 1961b and 1961c will reflect off reflecting surface 1916 which is angled at angle 1962b relative to path direction 1960b. The light pattern exiting reflecting surface 1916 travels along path direction 1956c towards reflecting surface 1936 and is indicated by bounding rays 1955c and 1955d. The light pattern indicated by 1955c and 1955d will reflect off reflecting surface 1936 which is angled at angle 1958c relative to path direction 1956c. The light pattern exiting reflecting surface 1936 travels along path direction 1960c towards reflecting surface 1918 and is indicated by bounding rays 1961c and 1961d. The light pattern indicated by 1961c and 1961d will reflect off reflecting surface 1918 which is angled at angle 1962a relative to path direction 1960c. The light pattern exiting reflecting surface 1918 travels along path direction 1964 towards reflecting surface 1934 and is indicated by bounding rays 1966a and 1966b.

Reflecting surface 1934 is positioned along and perpendicular to path 1964 and the light pattern indicated by bound rays 1966a and 1966b will reflect off reflecting surface 1934, reversing direction. Because the light pattern reflected off of reflecting surface 1934 travels in the opposite direction of 1964, it will reflect off reflectors 1918, 1936, 1916, 1938, 1914 and 1932 in a reverse fashion to the forward path until it arrives back at reflecting surface 1912 and reflects back towards light source 1950 at focal point 1918.

In FIG. 19 the reflectors 1912, 1914, 1916, 1918, 1932, 1938, 1936, and 1934 are shown integrated as part of reflecting assemblies 1910 and 1930, but in some embodiments the reflecting assembly and reflectors may be separate components. In some embodiments, the end reflector 1934 may not be included in the system such that the light pattern does not travel the reverse path. In some embodiments, the successive light patterns indicated by 1955a and 1955b, 1961a and 1961b, and 1966a and 1966b are drawn as completely parallel to the axis of travel. However, as discussed previously, light pattern indicated by 1955a and 1955b may exit reflector 1912 with non-zero divergence and the successive reflected light patterns may maintain this divergence. Some of the light may be lost at each reflection interface to absorption at the reflecting surface, to diffuse reflections, and/or to a portion of the light not being incident on the reflecting surfaces.

It may be desirable to configure a system to maximize the amount of light that passes to each successive reflection. In FIG. 19 the reflecting surfaces 1932, 1914, 1938, 1916, 1936, 1918, and 1934 are described as planar in nature as is desired in some embodiments. In some embodiments, these reflecting surfaces may be non-planar in nature and have additional effect on the reflected light such as increasing or decreasing divergence.

It should be appreciated that the light exiting reflecting surface 1912 in reflecting system 1900 may traverse the volume between reflecting assembly 1910 and 1930 up to fourteen times, thereby maximizing the spherical irradiance achieved by light pattern indicated by 1955 and 1955b. A portion of the light pattern indicated by 1955a and 1955b will travel along path 1956a, 1960a, 1956b, 1960b, 1956c, 1960c, and 1934 two times each, once on the path towards end reflector 1934, and once on the return path back to reflector 1912. Reflecting system 1800 and reflecting system 1900 operate according to similar principals but differ according to the number of reflecting surface and number of traversing path directions travelled by the light exiting reflecting surface 1912. It should be appreciated that the number of reflecting surfaces and the number of traversing path directions may be increased or decreased relative to system 1800 and 1900 in a specific embodiment and still operate according to the same principals disclosed herein.

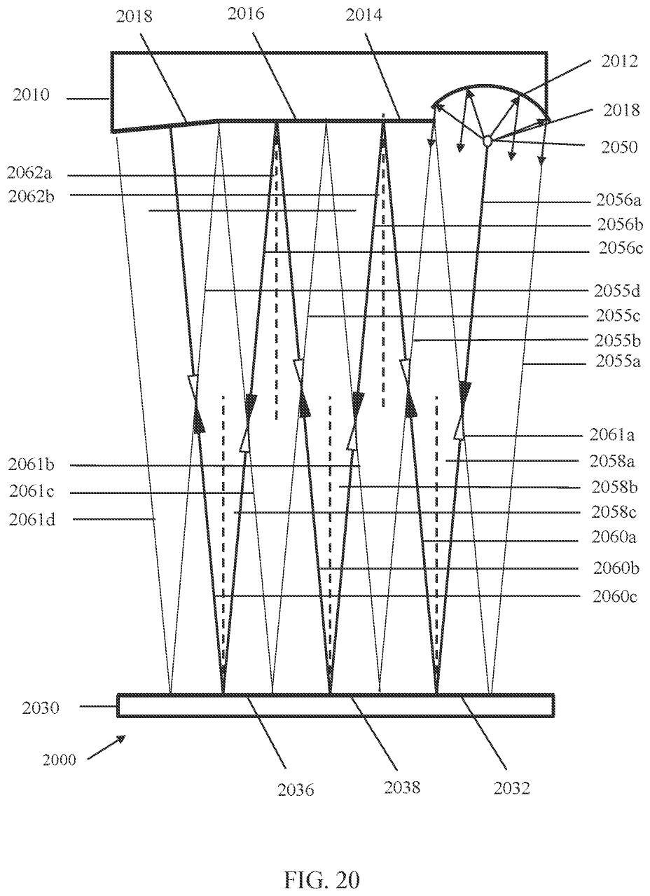

FIG. 20 is a schematic section view of a fourth embodiment of a reflecting system, similar to those described in FIG. 18 and FIG. 19 and indicated generally at 2000. Reflecting system 2000 includes a first reflecting assembly 2010, and a second reflecting assembly 2030. Reflecting assembly 2010 includes a paraboloid reflecting surface 2012, a light source 2050 located at the focal point 2018 of reflecting surface 2012, a second reflecting surface 2014, a third reflecting surface 2016, and a fourth reflecting surface 2018. Reflecting assembly 2030 includes a first reflecting surface 2032, a second reflecting surface 2038, and a third reflecting surface 2036.

In operation a light pattern exits reflecting surface 2012 as indicated by bounding rays 2055a and 2055b and travels along path direction 2056a towards reflecting surface 2032. Reflecting surface 2032 is position at angle 2058a relative to path direction 2056a and reflects the light. The light pattern exiting reflecting surface 2032 travels along path direction 2060a towards reflecting surface 2014 and is indicated by bounding rays 2061a and 2061b. The light pattern indicated by 2061a and 2062b will reflect off reflecting surface 2014 which is angled at angle 2062b relative to path direction 2060a. The light pattern exiting reflecting surface 2014 travels along path direction 2056b towards reflecting surface 2038 and is indicated by bounding rays 2055b and 2055c. The light pattern indicated by 2055b and 2055c will reflect off reflecting surface 2038 which is angled at angle 2058b relative to path direction 2056b. The light pattern exiting reflecting surface 2038 travels along path direction 2060b towards reflecting surface 2016 and is indicated by bounding rays 2061b and 2061c. The light pattern indicated by 2061b and 2061c will reflect off reflecting surface 2016 which is angled at angle 2062a relative to path direction 2060b. The light pattern exiting reflecting surface 2016 travels along path direction 2056c towards reflecting surface 2036 and is indicated by bounding rays 2055c and 2055d. The light pattern indicated by 2055c and 2055d will reflect off reflecting surface 2036 which is angled at angle 2058c relative to path direction 2056c. The light pattern exiting reflecting surface 2036 travels along path direction 2060c towards reflecting surface 2018 and is indicated by bounding rays 2061c and 2061d.

Reflecting surface 2018 is positioned along and perpendicular to path 2060c and the light pattern indicated by bound rays 2061c and 2061d will reflect off reflecting surface 2018, reversing direction. Because the light pattern reflected off of reflecting surface 2018 travels in the opposite direction of 2060c, it will reflect off reflectors 2036, 2016, 2038, 2014 and 2032 in a reverse fashion to the forward path until it arrives back at reflecting surface 2012 and reflects back towards light source 2050 at focal point 2018.

In FIG. 20 the reflectors 2012, 2014, 2016, 2018, 2032, 2038, and 2036 are shown integrated as part of reflecting assemblies 2010 and 2030, but in some embodiments the reflecting assembly and reflectors may be separate components. In some embodiments, the end reflector 2018 may not be included in the system such that the light pattern does not travel the reverse path. In some embodiments, the successive light patterns indicated by 2055a and 2055b, 2061a and 2061b, and 2066a and 2066b are drawn as completely parallel to the axis of travel. However, as discussed previously, light pattern indicated by 2055a and 2055b may exit reflector 2012 with non-zero divergence and the successive reflected light patterns may maintain this divergence. Some of the light may be lost at each reflection interface to absorption at the reflecting surface, to diffuse reflections, and/or to a portion of the light not being incident on the reflecting surfaces.

It may be desirable to configure a system to maximize the amount of light that passes to each successive reflection. In FIG. 20, the reflecting surfaces 2032, 2014, 2038, 2016, 2036, and 2018 are described as planar in nature as is desired in some embodiments. In some embodiments, these reflecting surfaces may be non-planar in nature and have additional effect on the reflected light such as increasing or decreasing divergence.

It should be appreciated that the light exiting reflecting surface 2012 in reflecting system 2000 may traverse the volume between reflecting assembly 2010 and 2030 up to twelve times, thereby maximizing the spherical irradiance achieved by light pattern indicated by 2055a and 2055b. A portion of the light pattern indicated by 2055a and 2055b will travel along path 2056a, 2060a, 2056b, 2060b, 2056c, and 2060c two times each, once on the path towards end reflector 2018, and once on the return path back to reflector 2012. It should be appreciated that the number of reflecting surfaces and the number of traversing path directions may be increased or decreased relative to system 2000 in a specific embodiment and still operate according to the same principals disclosed herein.

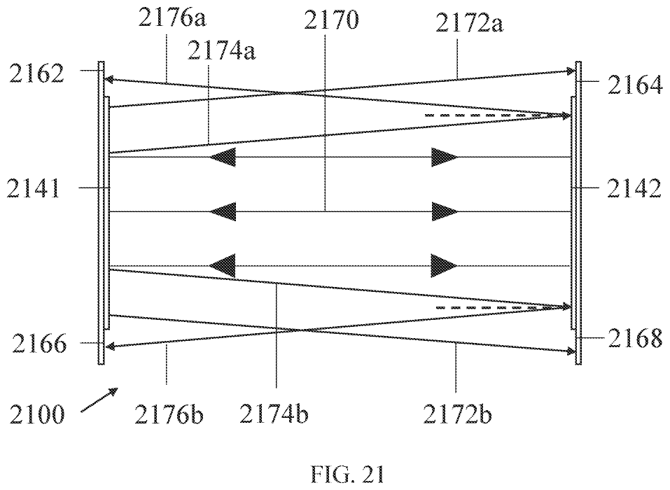

FIG. 21 shows a simplified side view of the second through fourth systems described in FIGS. 18, 19, and 20. While FIGS. 18, 19, and 20 are top views of a reflecting system, with light traveling between reflecting assemblies on the top and bottom of the figures, FIG. 21 is a side view with light travelling between reflecting assemblies on the left and right side of the figure. The reflectors are shown generally at 2141 and 2142. Absorbing borders 2162, 2164, 2166, and 2168 are also shown above and below the reflectors. These sections have a surface that will absorb a large portion of the light that is incident to them and will preferably reflect less than 10% of the incident light. The reflected light patterns are shown generally at 2170. Also shown are a select number of diverging rays indicated at 2172a, 2172b, 2174a, and 2174b. Ray 2172a exits reflector 2141 and strikes absorbing border 2164 where it is absorbed. Ray 2174a exits reflector 2141 and strikes reflector 2142 where is it reflected and this reflected ray 2176a then strikes absorbing border 2162 where it is absorbed. Ray 2172b exits reflector 2141 and strikes absorbing border 2168 where it is absorbed. Ray 2174b exits reflector 2141 and strikes reflector 2142 where is it reflected and this reflected ray 2176b then strikes absorbing border 2166 where it is absorbed. It should be appreciated that the absorbing borders 2162, 2164, 2166, and 2168 may prevent divergent rays from exiting the system 2100 which may eliminate or limit the light from entering the surrounding space.

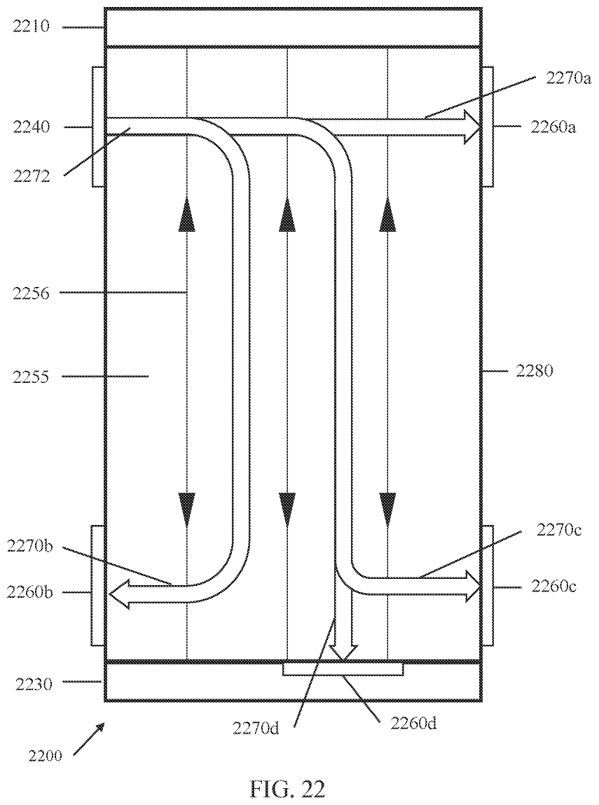

FIG. 22 shows a schematic section view of a fifth reflecting system, indicated generally at 2200. Reflecting system 2200 includes a first reflecting assembly 2210, a second reflecting assembly 2230, a plenum 2280, one or more fluid inlets 2240, and one or more fluid exits 2260a, 2260b, 2260c, and 2260d.

In operation, one or more light patterns 2256 will propagate between reflecting assemblies 2210 and 2230 in according with the principals described in other embodiments. A fluid 2272 such as air or water enters plenum 2280 at inlet 2240 under pneumatic pressure, hydrostatic pressure, or some other motive force and travels some path 2270a, 2270b, 2270c, or 2270d to some exit 2260a, 2260b, 2260c, or 2260d where it exits plenum 2280. Paths 2270a, 2270b, 2270c, or 2270d are shown schematically while in reality the bulk fluid travelling through plenum 2280 will have a complex nature with many different flows paths that are variable in nature. The fluid 2272 may be exposed to spherical irradiance within light pattern 2255 and may receive some cumulative dose of irradiation as it passes through plenum 2280. As used herein, dose refers to irradiance over time, and the dose received by a particle of fluid 2272 as it passes through plenum 2280 will equal the irradiance experience by the particle as it passes through 3D space multiplied by the time over which the particle is exposed to the irradiance. Note that the irradiance experienced at each point in space along the path travels by a particle of fluid 2272 will vary as the particle travels through a variable irradiance field and the time that the particle is present in each point in 3D space is dependent on path trajectory and velocity. In some cases, It may be desirable to use a time integral to evaluate the dose experience by fluid 2272.

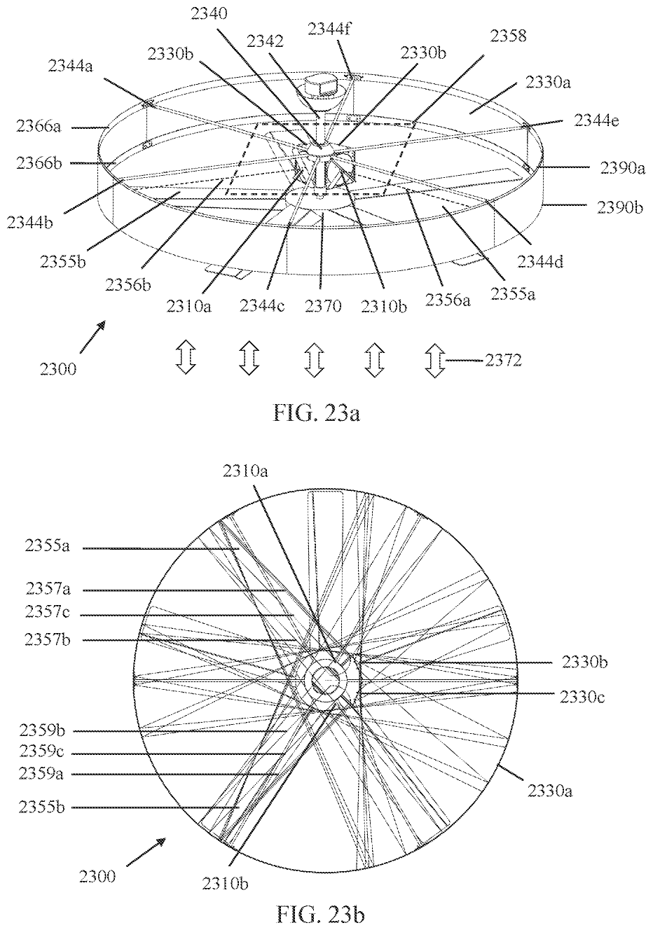

FIG. 23a is a side view of a sixth embodiment of a reflecting system, indicated generally at 2300. Reflecting system 2300 includes one or more reflecting assemblies 2310a, and 2310b that emit functional light in low divergence light patterns 2355a and 2255b travelling along path directions 2356a, and 2356b in accordance with the principles described above. Path directions 2356a, and 2356b are parallel to a reference plane 2358. Reflecting system 2300 also includes one or more reflecting surfaces 2330a, 2330b, 2330c that are perpendicular to reference plane 2358, as well as one or more absorbing surfaces 2366a and 2366b positioned proximate to one or more reflecting surfaces 2330a, 2330b, 2330c. In some embodiments, reflecting assembly 2300 also includes a mounting structure 2340 that positions and locates the reflecting assemblies 2310a, 2310b, and reflecting surfaces 2230a, 2230b, 2330c. In some embodiments mounting structure 2340 may include one or more central mounting elements 2342, and one or more secondary mounting elements 2344a, 2344b, 2344c, 2344d, 2344e, and 2344f. In some embodiments, reflecting system 2300 may include or be configured along the flow path of an air moving device 2370 that is configured to pass fluid such as air or water across light patterns 2355a and 2355b in a direction perpendicular to reference plane 2358.

In some embodiments, air moving device 2370 is selected from the group including axial fans, tube-axial fans, centrifugal fans, tesla turbines, and the like. Air moving device 2370 is preferably selected to pass fluid throughout the reflecting systems 2300 in an efficient and effective manner such that the fluid is exposed to and receives a functional dose of irradiation.

In operation, light patterns 2355a and 2355b will exit reflector assemblies 2310a, and 2310b along path directions 2356a and 2356b towards reflecting surface 2330a where it will reflect. Because reflecting surface 2330a is perpendicular to reference plane 2358, light patterns 2355a and 2355b will maintain their orientation parallel to reference plane 2358. Light patterns 2355a and 2355b may reflect off reflecting surface 2330a into one or more different directions parallel to reference plane 2358. These reflected rays will continue to propagate parallel to reference plane 2358. Some reflected rays will contact reflecting surface 2330a again at which point they will reflect a second time, remaining parallel to reference plane 2358. Light rays will continue to reflect off reflecting surfaces 2330a, 2330b, and 2330c until they are completely attenuated by the incremental loss of power at each reflection or by being absorbed by components within reflecting system 2300, and/or by exiting reflecting system 2300 to the surrounding environment.

As described in previous embodiments, the light patterns 2355a and 2355b may travel along path directions 2356a and 2356b, but also include some portion of divergent light rays with a direction at some non-zero angle to reference plane 2358. As these individual rays travel across the reflecting system, they will propagate some amount in a direction perpendicular to reference plane 2358. In some embodiments, the height or reflecting surfaces 2330a, 2330b, and 2330c are set to capture a sufficient portion of the light pattern exiting reflector assemblies 2310a, and 2310b while reasonably constraining the overall height of the reflecting surfaces 2330a, 2330b, and 2330c. For example, reflecting surfaces 2330a, 2330b, and 2530c may be limited to the height of the aperture of reflector assemblies 2310a and 2310b plus some additional height to account for some nominal amount of divergence. At some number of reflections, this propagation of diverging rays perpendicular to reference plane 2358 will result in the reflected rays not landing on reflecting surfaces 2330a, 2330b, and 2330c. In some embodiments, absorbing borders 2366a and 2366b may be sized such that some of the divergent light rays exiting reflecting assemblies 2310a, 2310b, or reflecting surfaces 2330a, 2330b, 2330c will contact absorbing borders 2360a and 2360b instead of exiting reflecting system 2300 and entering the surround environment. In some embodiments absorbing borders 2366a and 2366b may be sized such that some small amount such as e.g., less than 15% of the light exiting reflector assemblies 2310a and 2310b exit reflecting system 2300.

It should be appreciated that reflecting system 2300 enables the light patterns 2355a and 2355b exiting reflecting assemblies 2310a and 2310b to travel a relatively long path length by traversing across reflecting surface 2330a multiple times, while containing the light within reflecting system 2300 and limiting or eliminating light lost to the surrounding environment. Furthermore, the configuration described does not require physical barriers parallel to reference plane 2358 to contain the light, leaving the volume contained by reflecting surface 2330a open for unrestricted fluid flow driven by air moving device 2370. Air moving device 2370 drives fluid flow 2372 across the volume contained by reflecting surface 2330a where the fluid flow 2372 will receive some functional dose of irradiance as it crosses the reflected light patterns 2355a, 2355b and associated reflections.

In some embodiments reflecting system 2300 may include one or more light sources 2390a, 2390b in the visible, UV, or IR spectrum, positioned to have emission patterns exiting reflecting system 2300 to the surrounding environment for the purpose of space illumination, indication of device functionality, functional treatment of the surrounding environment, or for general indication of the status of the volume in which reflecting system 2300 is installed. For example, a color indicator may display a certain color pattern to show that the device is functionally correctly and active, a different color if functioning correctly and inactive, and yet another color if not functioning correctly. In other embodiments, the colors displayed may be consistent or time variant. In another example, light sources 2390a, and 2390b may be configured to indicate some room characteristic not directly related to the function of reflecting system 2300, such as air quality.

FIG. 23b is a top view of reflecting system 2300. Light pattern 2355a exiting reflector assembly 2310a is indicated by bounding rays 2357a, 2357b, and central ray 2357c. Light pattern 2355b exiting reflector assembly 2310b is indicated by bounding rays 2359a, 2359b, and central ray 2359c. The path of these rays parallel to reference plane 2358 are shown as they reflect off reflecting surfaces 2330a, 2330b, and 2330c. Accordingly, the long path length achieved by reflecting system 2300 may be visualized as a series of multi-reflection paths followed by the bounding and central rays.

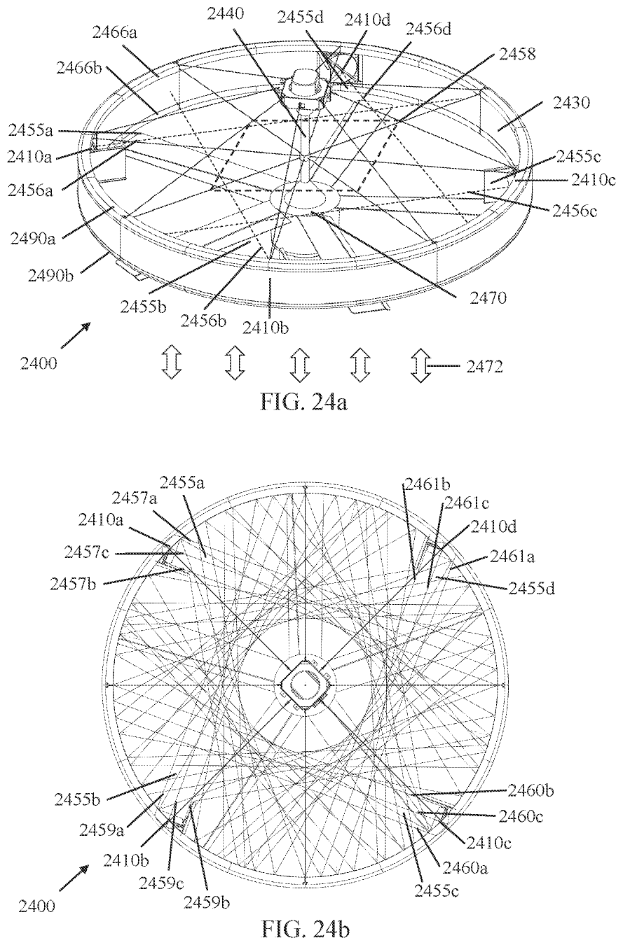

FIG. 24a is a side view of a seventh embodiment of a reflecting system, indicated generally at 2400. Reflecting system 2400 includes one or more reflecting assemblies 2410a, 2410b, 2410c, and 2410d that emit functional light in low divergence light patterns 2455a, 2455b, 2455c, and 2455d travelling along path directions 2456a, 2456b, 2456c and 2456d in accordance with the principles described above. Path directions 2456a, 2456b, 2456c and 2356d may be parallel to a reference plane 2458. Reflecting system 2400 also includes one or more reflecting surfaces 2430 that are perpendicular to reference plane 2458, as well as one or more absorbing surfaces 2466a and 2466b positioned proximate to one or more reflecting surfaces 2430. In some embodiments, reflecting assembly 2400 also includes a mounting structure 2440 that positions and locates the reflecting assemblies 2410a, 2410b, 2410c, 2410d and reflecting surfaces 2430. In some embodiments, reflecting system 2400 may include or be configured along the flow path of an air moving device 2470 that is configured to pass fluid such as air or water across light patterns 2455a, 2455b, 2455c, and 2455d in a direction perpendicular to reference plane 2458.

In operation, light patterns 2455a, 2455b, 2455c, and 2455d will exit reflector assemblies 2410a, 2410b, 2410c, and 2410d along path directions 2456a, 2456b, 2456c, and 2456d towards reflecting surface 2430 where it will reflect. Because reflecting surface 2430 is perpendicular to reference plane 2458, light patterns 2455a, 2455b, 2455c and 2455d will maintain their orientation parallel to reference plane 2458. Light patterns 2455a, 2355b, 2455c, and 2455d may reflect off reflecting surface 2430 into one or more different directions parallel to reference plane 2458. These reflected rays will continue to propagate parallel to reference plane 2458. Some reflected rays may contact reflecting surface 2430 again at which point they will reflect a second time, remaining parallel to reference plane 2458. Light rays may continue to reflect off reflecting surfaces 2430 until they are completely attenuated by the incremental loss of power at each reflection or by being absorbed by components within reflecting system 2400, and/or by exiting reflecting system 2400 to the surrounding environment.

As described in previous embodiments, the light patterns 2455a, 2455b, 2455c, and 2455d may travel along path directions 2456a, 2456b, 2456c and 2456d, but also include some portion of divergent light rays with a direction at some non-zero angle to reference plane 2458. In some embodiments, the height or reflecting surfaces 2430 is set to capture a sufficient portion of the light pattern exiting reflector assemblies 2410a, 2410b, 2410c, and 2410d while reasonably constraining the overall height of the reflecting surfaces 2430. For example, reflecting surface 2430 may be limited to the height of the aperture of reflector assemblies 2410a, 2410b, 2410c, and 2410d plus some additional height to account for some nominal amount of divergence. At some number of reflections, this propagation of diverging rays perpendicular to reference plane 2458 will result in the reflected rays not landing on reflecting surface 2430. In some embodiments, absorbing borders 2466a and 2466b may be sized such that the divergent light rays exiting reflecting assemblies 2410a, 2410b, 2410c, 2410d or reflecting surface 2430 will contact absorbing borders 2466a and 2466b instead of exiting reflecting system 2400 and entering the surrounding environment. In some embodiments absorbing borders 2466a and 2466b may be selected such that some small amount such as less than 15% of the light exiting reflector assemblies 2410a, 2410b, 2410c, and 2410d exit reflecting system 2400.

It should be appreciated that reflecting system 2400 enables the light patterns 2455a, 2455b, 2455c, and 2455d exiting reflecting assemblies 2410a, 2410b, 2410c, and 2410d to travel a relatively long path length by traversing across reflecting surface 2430 multiple times, while containing the light within reflecting system 2400 and limiting or eliminating light lost to the surrounding environment. Furthermore, the configuration described does not require physical barriers parallel to reference plane 2458 to contain the light, leaving the volume contained by reflecting surface 2430 open for unrestricted fluid flow driven by air moving device 2470. Air moving device 2470 drives fluid flow 2472 across the volume contained by reflecting surface 2430 where the fluid flow 2472 will receive some functional dose of irradiance as it crosses the reflected light patterns 2455a, 2455b, 2455c, and 2455d and associated reflections. Similar to air moving device 2370, air moving device 2470 may be selected from the group including axial fans, tube-axial fans, centrifugal fans, tesla turbines, and the like.

In some embodiments reflecting system 2400 may include one or more light sources 2490a, 2490b in the visible spectrum positioned to have emission patterns exiting reflecting system 2400 to the surrounding environment for the purpose of space illumination, indication of device functionality, functional treatment of the surrounding environment, or for general indication. For example, a color indicator may display a certain color pattern to show that the device is functionally correctly and active, a different color if functioning correctly and inactive, and yet another color if not functioning correctly. In other embodiments, the colors displayed may be consistent or time variant. In another example, light sources 2490a, and 2490b may be configured to a indicate some room characteristic not directly related to the function of reflecting system 2400, such as air quality.

FIG. 24b is a top view of reflecting system 2400. Light pattern 2455a exiting reflector assembly 2410a is indicated by bounding rays 2457a, 2457b, and central ray 2457c. Light pattern 2455b exiting reflector assembly 2410b is indicated by bounding rays 2459a, 2459b, and central ray 2459c. Light pattern 2455c exiting reflector assembly 2410c is indicated by bounding rays 2460a, 2460b, and central ray 2460c. Light pattern 2455d exiting reflector assembly 2410d is indicated by bounding rays 2461a, 2461b, and central ray 2461c. The path of these rays parallel to reference plane 2458 are shown as they reflect off reflecting surfaces 2430. Accordingly, the long path length achieved by reflecting system 2400 may be visualized as a series of multi-reflection paths followed by the bounding and central rays.

As described above, the term long path is used herein to describe an optical system where the light source travels a distance of two or more times the bounding dimension of the volume in which the light source operates.

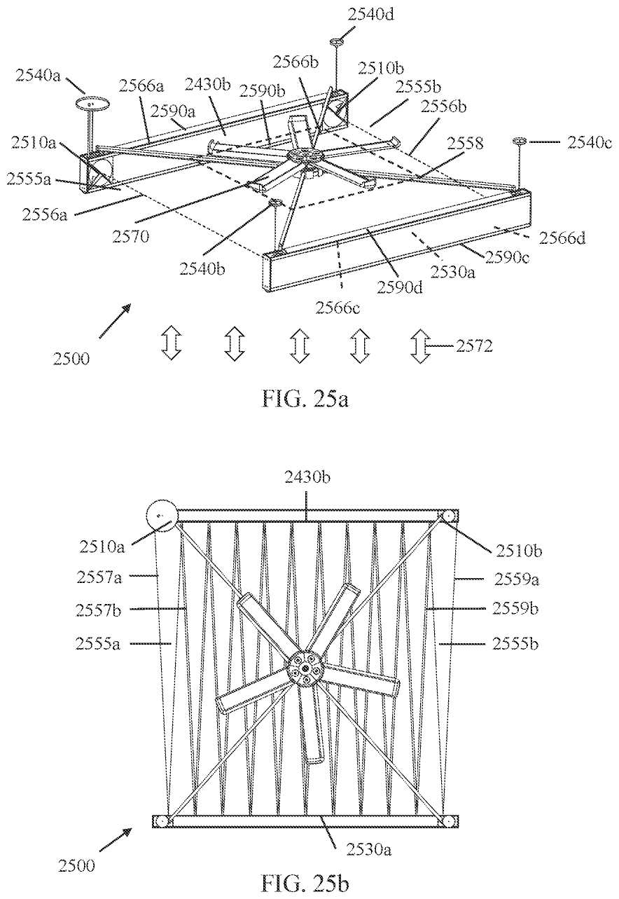

FIG. 25a shows a side view of an eight embodiment of a reflecting system, indicated generally at 2500. Reflecting system 2500 includes one or more reflecting assemblies 2510a, and 2510b that emit functional light in low divergence light patterns 2555a and 2555b travelling along path directions 2556a, and 2556b in accordance with the principles described above. Path directions 2556a, and 2556b are parallel to a reference plane 2558. Reflecting system 2500 also includes one or more reflecting surfaces 2530a and 2530b that are perpendicular to reference plane 2558, as well as one or more absorbing surfaces 2566a, 2566b, 2566c, and 2566d positioned proximate to one or more reflecting surfaces 2530a, 2530b. In some embodiments, reflecting assembly 2500 also includes mounting structures 2540a, 2540b, 2540c, and 2540d that position and locate the reflecting assemblies 2510a, 2510b, and reflecting surfaces 2530a, 2530b. In some embodiments, reflecting system 2500 may include an air moving device 2570 that is configured to pass fluid such as air or water across light patterns 2555a and 2555b in a direction perpendicular to reference plane 2558.

In operation, light patterns 2555a and 2555b will exit reflector assemblies 2510a, and 2510b along path directions 2556a and 2556b towards reflecting surface 2530a where they will reflect. Because reflecting surfaces 2530a and 2530b are perpendicular to reference plane 2558, light patterns 2555a and 2555b will maintain their orientation parallel to reference plane 2558. Light patterns 2555a and 2555b may reflect off reflecting surface 2530a into one or more different directions parallel to reference plane 2558. These reflected rays will continue to propagate towards reflecting surface 2530b parallel to reference plane 2558. Some reflected rays will contact reflecting surface 2530b at which point they will reflect a second time back towards reflecting surface 2530a, remaining parallel to reference plane 2558. Light rays may continue to reflect off reflecting surfaces 2530a, and 2530c until they are completely attenuated by the incremental loss of power at each reflection or by being absorbed by components within reflecting system 2500, and/or by exiting reflecting system 2500 to the surrounding environment.

As described in previous embodiments, the light patterns 2555a and 2555b may travel along path directions 2556a and 2556b, but also include some portion of divergent light rays with a direction at some non-zero angle to reference plane 2558. As these individual rays travel across the reflecting system, they will propagate some amount in a direction perpendicular to reference plane 2558. In some embodiments, the height or reflecting surfaces 2530a and 2530b are set to capture a sufficient portion of the light pattern exiting reflector assemblies 2510a, and 2510b while reasonably constraining the overall height of the reflecting surfaces 2530a and 2530b. For example, reflecting surfaces 2530a and 2530b may be limited to the height of the aperture of reflector assemblies 2510a and 2510b plus some additional height to account for some nominal amount of divergence. At some number of reflections, this propagation of diverging rays perpendicular to reference plane 2558 will result in the reflected rays not landing on reflecting surfaces 2530a and 2530b. In some embodiments, absorbing borders 2566a, 2566b, 2566c, and 2566d may be selected such that the divergent light rays exiting reflecting assemblies 2510a, 2510b, or reflecting surfaces 2530a, 2530b will contact absorbing borders 2566a, 2566b, 2566c, and 2566d instead of exiting reflecting system 2500 and entering the surround environment. In some embodiments absorbing borders 2566a, 2566b, 2566c and 2566d may be selected such that some small amount, such as less than 15% of the light exiting reflector assemblies 2510a and 2510b, exit reflecting system 2500.

It may be appreciated that reflecting system 2500 enables the light patterns 2555a and 2555b exiting reflecting assemblies 2510a and 2510b to travel a relatively long path length by traversing between reflecting surfaces 2530a and 2530b multiple times, while containing the light within reflecting system 2500 and limiting or eliminating light lost to the surrounding environment. Furthermore, the configuration described does not require physical barriers parallel to reference plane 2558 to contain the light, leaving the volume contained by reflecting surface 2530a and 2530b open for unrestricted fluid flow driven by air moving device 2570. As is appreciated from other embodiments, air moving device 2570 drives fluid flow 2572 across the volume contained by reflecting surfaces 2530a and 2530b where the fluid flow 2572 will receive some functional dose of irradiance as it crosses the reflected light patterns 2555a, 2555b and associated reflections.

In some embodiments reflecting system 2500 may include one or more light sources 2590a, 2590b, 2590c, and 2590d in the visible spectrum positioned to have emission patterns exiting reflecting system 2500 to the surrounding environment for the purpose of space illumination, indication of device functionality, functional treatment of the surrounding environment, or for general indication. For example, a color indicator may display a certain color pattern to show that the device is functionally correctly and active, a different color if functioning correctly and inactive, and yet another color if not functioning correctly. In other embodiments, the colors displayed may be consistent or time variant. In another example, light sources 2590a, 2590b, 2590c, and 2590d may be configured to a indicate some room characteristic not directly related to the function of reflecting system 2500, such as air quality.

FIG. 25b is a top view of reflecting system 2500. Light pattern 2555a exiting reflector assembly 2510a is indicated by bounding rays 2557a and 2557b. Light pattern 2555b exiting reflector assembly 2510b is indicated by bounding rays 2559a, and 2559b. The path of these rays parallel to reference plane 2558 are shown as they reflect between reflecting surfaces 2530a and 2530b. Accordingly, the long path length achieved by reflecting system 2500 may be visualized as a series of multi-reflection paths followed by the bounding and central rays.

Reflecting systems disclosed herein may effectively disinfect fluids and may be manufactured and sold at a lower cost than commercially available irradiation systems. They may be small enough to fit wherever needed and be conveniently movable from one location to another. While use cases specific to disinfection of air or water have been described, the reflecting systems disclosed may also be applied in applications where a light pattern with adjustable direction and divergence are beneficial, such as in agriculture where delivering tightly controlled light to a specific locations allows for targeted delivery of light to specific crops. Or in UV curing applications where targeted delivery of light to a surface may be desirable.

The above description of the disclosed embodiments is provided to enable any person skilled in the art to make or use the disclosure. Various modifications to these embodiments will be readily apparent to those skilled in the art, and the generic principles described herein can be applied to other embodiments without departing from the spirit or scope of the disclosure. For example, while the light sources have been generally described as fixed, a system that is capable of adjusting the position of a light source relative to the focal point of a reflector, or capable of adjusting the position or shape of a reflector in real time may operate adaptively, adjusting to the needs of a room and situation according to some optimized behavior in response to one or more sensors or commands.

Thus, it is to be understood that the description and drawings presented herein represent exemplary embodiments of the disclosure and are therefore representative of the subject matter, which is broadly contemplated by the present disclosure. It is further understood that the scope of the present disclosure fully encompasses other embodiments and that the scope of the present disclosure is accordingly limited by nothing other than the appended claims.

1. A device for controlling the direction of light from a light source comprising: a first reflector having a first focal point;
a light source positioned proximate to the first focal point of the first reflector;
wherein the light source provides light to the first reflector from a first position having a beam angle of 180 degrees or less and wherein the first reflector reflects the light in two or more substantially collimated rays such that the two or more collimated rays are substantially parallel to each other in a first output pattern.

2.-27. (canceled)

28. The device of claim 1, wherein the light source is movable along an axis relative to the first reflector, wherein moving the light source to a second position results in a controlled degree of divergence of the reflected light pattern.

29. The device of claim 28, wherein the divergence is less than approximately 10 degrees.

30. The device of claim 29, wherein the light source movement is automated.

31.-33. (canceled)

34. The device of claim 1, further comprising a second reflector, wherein the second reflector receives the first output pattern from the first reflector and redirects a portion of the two or more collimated rays into a second output pattern.

35. The device of claim 34, wherein the second output pattern has a different central direction and/or divergent characteristics than the first output pattern.

36. The device of claim 34, wherein the second reflector has a shape that is adjustable.

37. A system comprising:
a printed circuit board; and
two or more of the device of claim 1 mounted to the printed circuit board.

38. A system for disinfecting a fluid comprising: at least one ultraviolet (UV) light source;
a first reflector having a first reflective surface;
a second reflector having a first reflective surface;
wherein the first and second reflectors are opposite each other and located a predetermined distance from each other, wherein the first reflector emits a first pattern of UV light having a divergence of less than 10 degrees, wherein the second reflect reflector emits a second pattern of UV light having a divergence of less than 10 degrees, the first pattern and second patterns being different; and
wherein at least a portion of the first light pattern travels across a volume two or more times the length of the predetermined distance between the first and second reflectors.

40. The system of claim 39, wherein the first reflector has a first focal point and the light source is positioned proximate to the first focal point of the first reflector.

41. The system of claim 39, wherein the system further comprises a fluid plenum and wherein the reflectors are located within the fluid plenum.

42. The system of claim 41, wherein the reflectors provide a functional dose of irradiation to the fluid via the pattern of UV light.

43. The system of claim 39, further comprising one or more absorbing borders are positioned proximal to reflecting surfaces.

44. A system for disinfecting a fluid comprising: a reflecting device having:
at least two reflective internal surfaces spaced a predetermined distance apart, and
one or more light sources emitting a pattern of ultraviolet (UV) light having a divergence of less than 10 degrees,
wherein at least a portion of the light pattern travels across from a first reflective internal surface to a second internal surface and back again, and
wherein fluid passing through the at least two reflective internal surface receives a functional dose of irradiance from exposure to the pattern of UV light.

45. The system of claim 44, where one or more absorbing borders are positioned proximate to said reflective internal surfaces.

46. The system of claim 44, wherein the reflecting device comprises a ring shape, the ring shape having a plurality of internal reflective surfaces that are perpendicular to the direction of the light source.

47. The system of claim 44, further comprising:
a fluid moving device that passes fluid through the reflecting device.

48. The system of claim 47, wherein the fluid moving device comprises an axial ceiling fan.

49. The system of claim 44, wherein the one or more light sources are positioned proximate to the at least two reflective internal surfaces of the reflecting device and wherein the light sources are directed towards the at least two reflective internal surfaces of the reflecting device.

50. The system of claim 49, wherein the one or more light sources are positioned in the center of the reflecting device or along a peripheral edge of the reflecting device.
